SEQUENCE LISTING (2 sequences)

SEQ ID NO: 1 - DNA, 7127 bp, Homo sapiens
ttcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttatgtatcatacacatacgatttaggtgacactatagaataacatccactttgcctttctctccacaggtgtccactcccaggtccaactgcacctcggttctatcgatatgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgcggggtcaccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccctagagtcgacctgcagaagcttctagagtcgacctgcagaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgatcgggaattaattcggcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgttaattcgaacacgcagatgcagtcggggcggcgcggtcccaggtccacttcgcatattaaggtgacgcgtgtggcctcgaacaccgagcgaccctgcagcgacccgcttaacagcgtcaacagcgtgccgcagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaacccccaagttcgggtgaaggcccagggctcgcagccaacgtcggggcggcaagcccgccatagccacgggccccgtgggttagggacggggtcccccatggggaatggtttatggttcgtgggggttattcttttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgccgccggacgaactaaacctgactacggcatctctgccccttcttcgctggtacgaggagcgcttttgttttgtattggtcaccacggccgagtttccgcgggaccccggccagggcacctgtcctacgagttgcatgataaagaagacagtcataagtgcggcgacgatagtcatgccccgcgcccaccggaaggagctgactgggttgaaggctctcaagggcatcggtcgagcggccgcatcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcaggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgatgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccagcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagctggcacgacaggtttcccgactggaaagcgggcagtgagcgcaacgcaattaatgtgagttacctcactcattaggcaccccaggctttacactttatgcttccggctcgtatgttgtgtggaattgtgagcggataacaatttcacacaggaaacagctatgaccatgattacgaattaa

SEQ ID NO: 2 - protein, 397 aa, Homo sapiens
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGVTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2006/0205660 A1
(43) Pub. Date: Sep. 14, 2006

(54) OB protein-immunoglobulin chimeras

(76) Inventors: Frederic De Sauvage, Foster City, CA (US); Nancy Levin, Newbury Park, CA (US); Richard L. Vandlen, Hillsborough, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/433,186

(22) Filed: May 11, 2006

Related U.S. Application Data
(63) Continuation of application No. 08/793,653, filed on Feb. 27, 1997.
(63) Continuation-in-part of application No. 08/667,184, filed on Jun. 20, 1996, now abandoned.

(51) Int. Cl. A61K 38/17; C07K 14/705; C07H 21/04; C12P 21/06

(52) U.S. Cl. 514/12; 435/69.1; 435/320.1; 435/325; 530/350; 536/23.5

(57) ABSTRACT

The present invention concerns long-half derivative of the obesity protein OB. The invention specifically concerns OB protein-immunoglobulin chimeras and polyethylene glycol (PEG)-OB derivatives, which have extended half-life as compared to the corresponding native OB proteins. The invention further relates to methods for appetite and/or weight reducion and for treating other physiological conditions by using the long half-life derivatives of OB.

### CROSS REFERENCES

This is a non-provisional application that is a continuation of U.S. application Ser. No. 08/793,653, filed Feb. 27, 1997, which is the U.S. national phase application of PCT/US96/20718, filed Dec. 19, 1996. U.S. Application 08/793,653 and the present application claim priority as a continuation-in-part of U.S. application Ser. No. 08/667,184, filed on Jun. 20, 1996, now abandoned. U.S. application Ser. No. 08/793,653 and the present application also claim priority to U.S. Provisional Application No. 60/040,911, filed on Dec. 27, 1995.

### FIELD OF THE INVENTION

The invention concerns long half-life derivatives of the OB protein. In particular, the invention concerns OB protein-immunoglobulin chimeras, and other long half-life derivatives of the OB protein, and compositions comprising and methods for administering them. The invention further relates to a method for treating obesity by administering a long half-life variant of the OB protein, such as, an OB protein-immunoglobulin chimera.

### BACKGROUND OF THE INVENTION

Obesity is the most common nutritional disorder which, according to recent epidemiologic studies, affects about one third of all Americans 20 years of age or older. Kuczmarski et al., J. Am. Med. Assoc. 272, 205-11 (1994). Obesity is responsible for a variety of serious health problems, including cardiovascular disorders, type II diabetes, insulin-resistance, hypertension, hypertriglyceridemia, dyslipoproteinemia, and some forms of cancer. Pi-Sunyer, F. X., Anns. Int. Med. 119,655-60 (1993); Colfitz, G. A., Am. J. Clin. Nutr. 55, 503S-507S (1992). A single-gene mutation (the obesity or “ob” mutation) has been shown to result in obesity and type II diabetes in mice. Friedman, Genomics 11, 1054-1062 (1991). Zhang et al., Nature 372,425-431(1994) have recently reported the cloning and sequencing of the mouse ob gene and its human homologue, and suggested that the ob gene product may function as part of a signalling pathway from adipose tissue that acts to regulate the size of the body fat depot. Parabiosis experiments performed more than 20 years ago predicted that the genetically obese mouse containing two mutant copies of the ob gene (ob/ob mouse) does not produce a satiety factor which regulates its food intake, while the diabetic (db/db) mouse produces but does not respond to a satiety factor. Coleman and Hummal, Am. J. Physiol. 217, 1298-1304 (1969); Coleman, Diabetol 9,294-98 (1973). Recent reports by three independent research teams have demonstrated that daily injections of recombinant OB protein inhibit food intake and reduce body weight and fat in grossly obese ob/ob mice but not in db/db mice (Pelleymounter et al., Science 269, 540-43 [1995]; Halaas et al., Science 269, 543-46 [1995]; Campfield et al., Science 269, 546-49 [1995]), suggesting that the ob protein is such a satiety factor as proposed in early cross-circulation studies. The results of these first studies leave many questions unanswered, and show a number of as yet unresolved discrepancies. For example, while modest effects of daily injections of the ob protein on food intake and body weight were reported in lean mice, there was a significant reduction in body fat as assessed by carcass composition in one (Halaas et al., supra) but not in another (Pelleymounter et al., supra) of these reports, despite equivalent decreases in body weight. Furthermore, Pelleymounter et al., supra observed that, for reasons unknown, ob/ob mice treated with a 0.1 mg/kg/day dose of the OB protein actually increased their body weight by 17.13%, while the weight reduction in the obese mice that received a 1 mg/kg/day dose of ob was rather moderate. The receptor or receptors of the ob protein are as of yet unidentified. While the existence of peripheral receptors cannot be ruled out at this time, the recent report that an increased expression of the ob gene in adipose tissue of mice with hypothalamic lesions does not result in a lean phenotype suggests that the OB protein does not act directly on fat cells. Maffei et al., Proc. Natl. Acad. Sci. 92, 6957-60 (1995). Researchers suggest that at least one OB receptor is localized in the brain. The identification and expression cloning of a leptin receptor (OB-R) was reported by Tartaglia et al., Cell 83, 1263-71 (1995). Various isoforms of a leptin receptor are described by Cioffi et al., Nature 2, 585-89 (1996). A human hematopoetin receptor, which might be a receptor of the OB protein, is described in PCT application Publication No. WO 96/08510, published 21 Mar. 1996. A receptor of the OB protein is disclosed in Tartaglia et al., Cell 83, 1263-71 (1995).

### SUMMARY OF THE INVENTION

The present invention is based on the observation that the OB protein is significantly more effective at reducing body weight and adipose tissue weight when delivered as a continuous subcutaneous infusion than when the same dose is delivered as a daily subcutaneous injection. The invention is further based on the unexpected finding that a chimeric protein, in which the OB polypeptide is fused to an immunoglobulin constant domain, is strikingly more potent in reducing the body weight and adipose depots than native human OB, when both proteins are administered by subcutaneous injection once a day. The latter observation is particularly surprising since the OB protein-immunoglobulin chimera due to its large molecular weight, is not expected to be able to cross the blood-brain barrier, and reach the OB receptor which has been believed to be located in the brain.

In one aspect, the invention concerns long half-life derivatives of an OB protein capable of reducing body weight and/or food intake in an individual treated. The invention further concerns compositions containing such derivatives, and their administration for reducing body weight and/or food intake.

In another aspect, the invention concerns chimeric polypeptides comprising an OB protein amino acid sequence capable of binding to a native OB receptor linked to an immunoglobulin sequence (briefly referred to as OB-immunoglobulin chimeras or immunoadhesins). In a specific embodiment, the chimeric polypeptides comprise a fusion of an OB amino acid sequence capable of binding a native OB receptor, to an immunoglobulin constant domain sequence. The OB portion of the chimeras of the present invention preferably has sufficient amino acid sequences from a native OB protein to retain the ability to bind to and signal through a native OB receptor. Most preferably, the OB protein retains the ability to reduce body weight when administered to obese human or non-human subjects. The OB polypeptide is preferably human, and the fusion is preferably with an immunoglobulin heavy chain constant domain sequence. In a particular embodiment, the association of two OB polypeptide-immunoglobulin heavy chain fusions (e.g., via covalent linkage by disulfide bond(s)) results in a homodimeric immunoglobulin-like structure. An immunoglobulin light chain may further be associated with one or both of the OB-immunoglobulin chimeras in the disulfide-bonded dimer to yield a homotrimeric or homotetrameric structure.

The invention further concerns nucleic acid encoding chimeric polypeptide chains of the present invention, expression vectors containing DNA encoding such molecules, transformed host cells, and methods for the production of the molecules by cultivating transformant host cells.

Although the long half-life derivatives of the present invention are particularly useful for reducing body weight and/or food intake, they can generally be used for the treatment of conditions associated with the abnormal expression or function of the OB gene and/or to elicit biological responses mediated by an OB receptor. Thus, the OB derivatives of the present invention may be used to treat bulemia, to reduce insulin levels, e.g. in Type I or II diabetic patients, and as mitogens of various cell types expressing an OB receptor. All these and related uses are within the scope of the present invention.

In another embodiment, the invention concerns the purification of an OB receptor by using an OB protein-immunoglobulin chimera.

### BRIEF DESCRIPTION OF THE FIGURES

FIG. 1 top—Lean female mice were treated with murine OB protein either as a continuous subcutaneous infusion or daily subcutaneous injections. The data shown are the mean body weight of each group, in grams, n=4 mice/point.

FIG. 1 bottom—The mean weight of the retroperitoneal fat pads are shown. Continuous subcutaneous infusions of the OB protein were also more effective than daily subcutaneous injections at reducing adipose tissue weight.

FIG. 2 top—Obese female ob/ob mice were treated with human OB protein (hOB) or with a human OB-IgG-1 fusion protein (hOB-IgG-1). The data shown are the mean change in body weight for each treatment group from the first to the last day of experiment, in grams, n=3 mice/bar except for the hOB 0.19 mg/kg/day by injection group, where n=4, and PBS injection group, where n=1.

FIG. 2 bottom—The data shown were the mean food intake for each treatment group for the six 24 hour periods of the experiment, in grams/mouse/day, n=1/bar.

FIG. 3 top and bottom—Obese (ob/ob) female mice were treated with either hOB or the hOB-IgG-1 fusion protein by daily subcutaneous injections for 7 days. The data are depicted as in FIG. 2, with n=4 for all treatment groups.

FIG. 4 top—Obese female ob/ob mice were treated with human protein (hOB) or with PEG-hOB. The data shown are the mean change in body weight for each treatment group from the first to the last day of experiment, in grams, n=3-4 mice/bar except for the PBS injection group, where n=1. The materials were injected daily subcutaneously. The “PEG 1X” and “PEG 2X” refer to the ratio of the PEG reagent to protein in the preparation of the molecule.

FIG. 4 bottom—The data shown were the mean food intake for each treatment group for the six 24 hour periods of the experiment, in grams/mouse/day, n=3-4/bar.

FIG. 5—Obese (ob/ob) female mice were treated with either the hOB-IgG fusion protein, native hOB, or hCD4-IgG by daily subcutaneous injections for 7 days, n=6 for all treatment groups, except hOB at 3.8 mg/kg/d, where n=2. Again it was observed that the fusion protein was more effective than the native hOB protein at reducing body weight (top and middle panels) and food intake (bottom panel).

FIG. 6—The nucleotide sequence (SEQ. ID. NO: 1) and the amino acid sequence (SEQ. ID. NO: 2) of the human OB-IgG-1 chimera of Example 1.

### DETAILED DESCRIPTION OF THE INVENTION

A. Definitions

The term “obesity” is used to designate a condition of being overweight associated with excessive bodily fat. The desirable weight for a certain individual depends on a number of factors including sex, height, age, overall built, etc. The same factors will determine when an individual is considered obese. The determination of an optimum body weight for a given individual is well within the skill of an ordinary physician.

The phrase “long half-life” and grammatical variants thereof, as used in connection with OB derivatives, concerns OB derivatives having a longer plasma half-life and/or slower clearance than a corresponding native OB protein. The long half-life derivatives preferably will have a half-life at least about 1.5-times longer than a native OB protein; more preferably at least about 2-times longer than a native OB protein, more preferably at least about 3-time longer than a native OB protein. The native OB protein preferably is that of the individual to be treated.

The terms “OB”, “OB polypeptide”, “OB protein” and their grammatical variants are used interchangeably and refer to “native” or “native sequence” OB proteins (also known as “leptins”) and their functional derivatives. The OB polypeptides have the typical structural features of cytokines, i.e. polypeptides released by one cell population which act on another cell as intercellular mediators, such as, for example, growth hormones, insulin-like growth factors, interleukins, insulin, glycoprotein hormones such as, follicle stimulating hormone (FSH), thyroid stimulating hormone (TSH), tumor necrosis factor-α and -β (TNF-α and -β), nerve growth factors, such as NGF-β, PDGF, transforming growth factors (TGFs) such as, TGF-α and TGF-β, insulin-like growth factor-1 and -2 (IGF-1 and IGF-2), erythropoietin, osteoinductive factors, interferons (IFNs) such as, IFN-α, IFN-β and IFN-γ, colony stimulating factors (CSFs) such as, M-CSF, GM-CSF, and G-CSF, interleukins (ILs) such as, IL-1, IL-2, IL-3, IL-4, IL-5, IL-6, IL-7, IL-8 and other polypeptide factors.

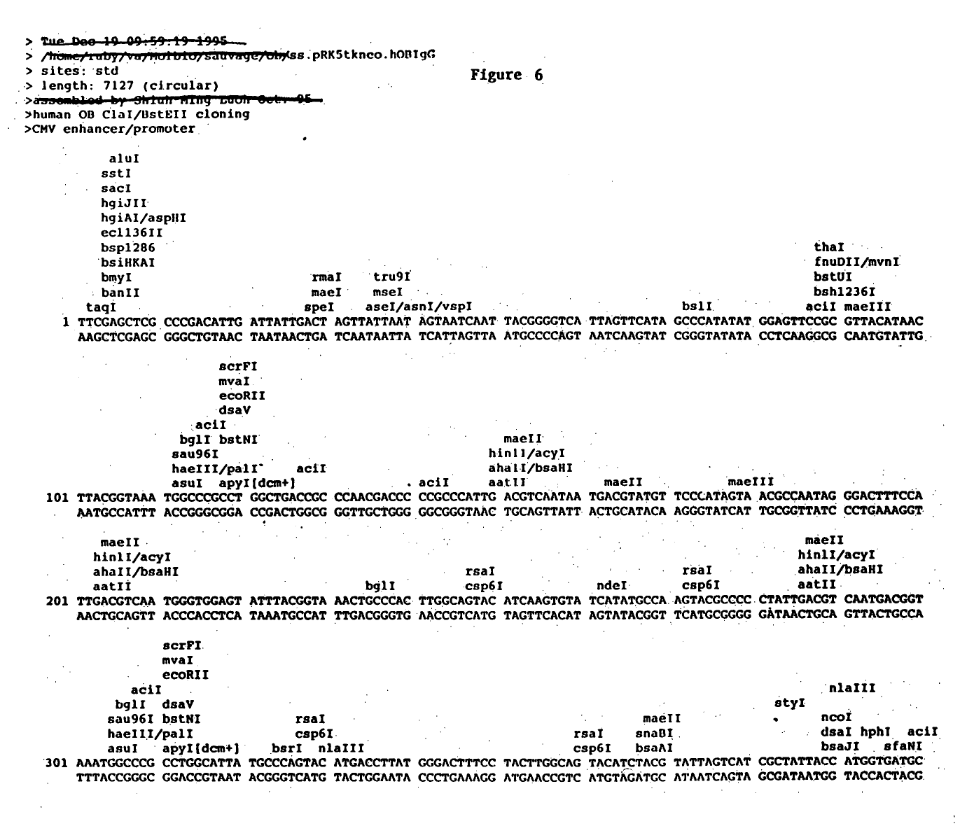

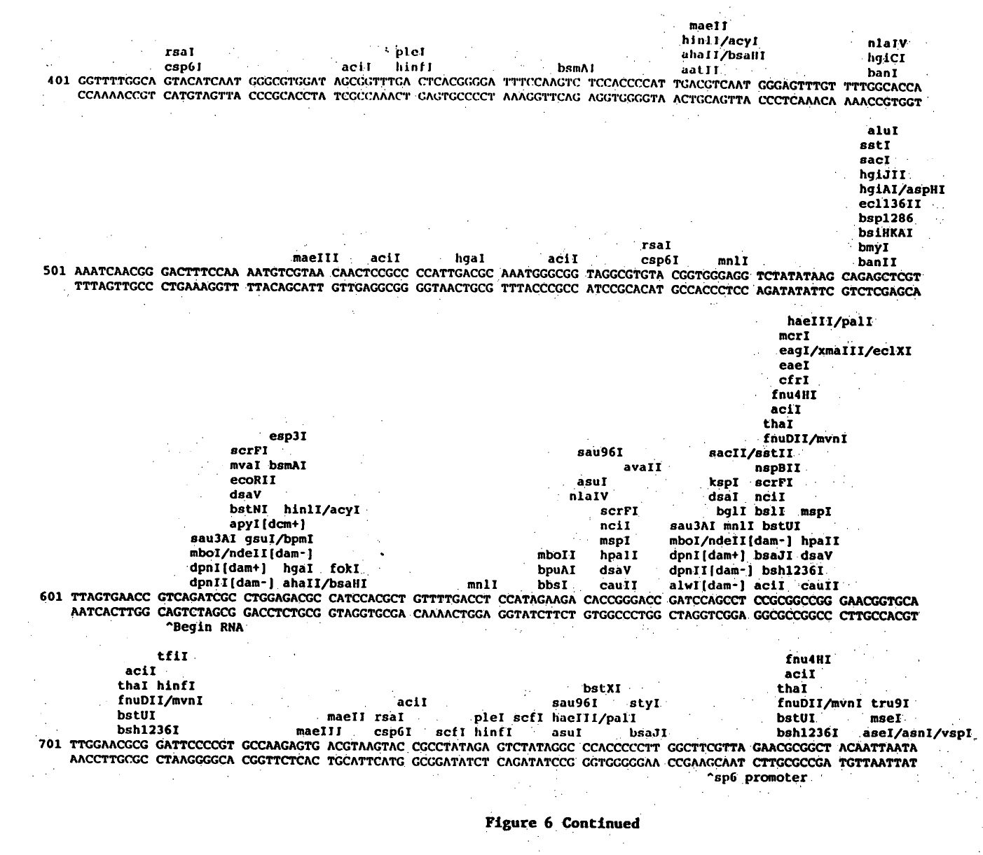

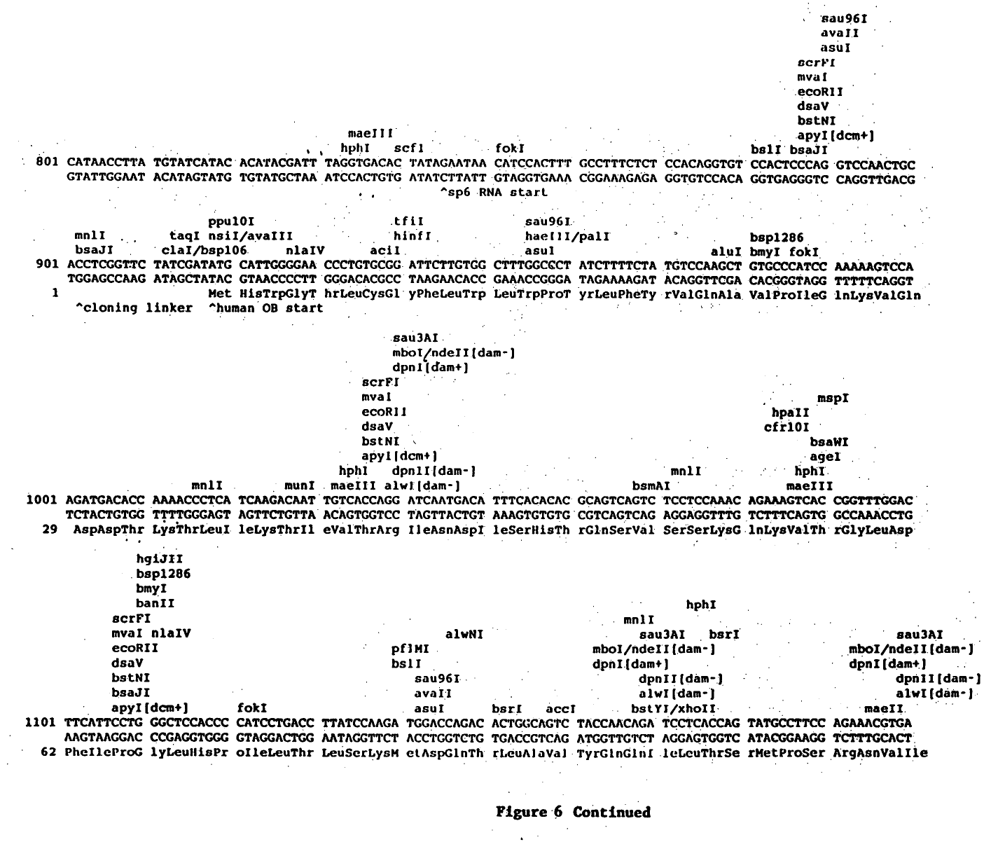

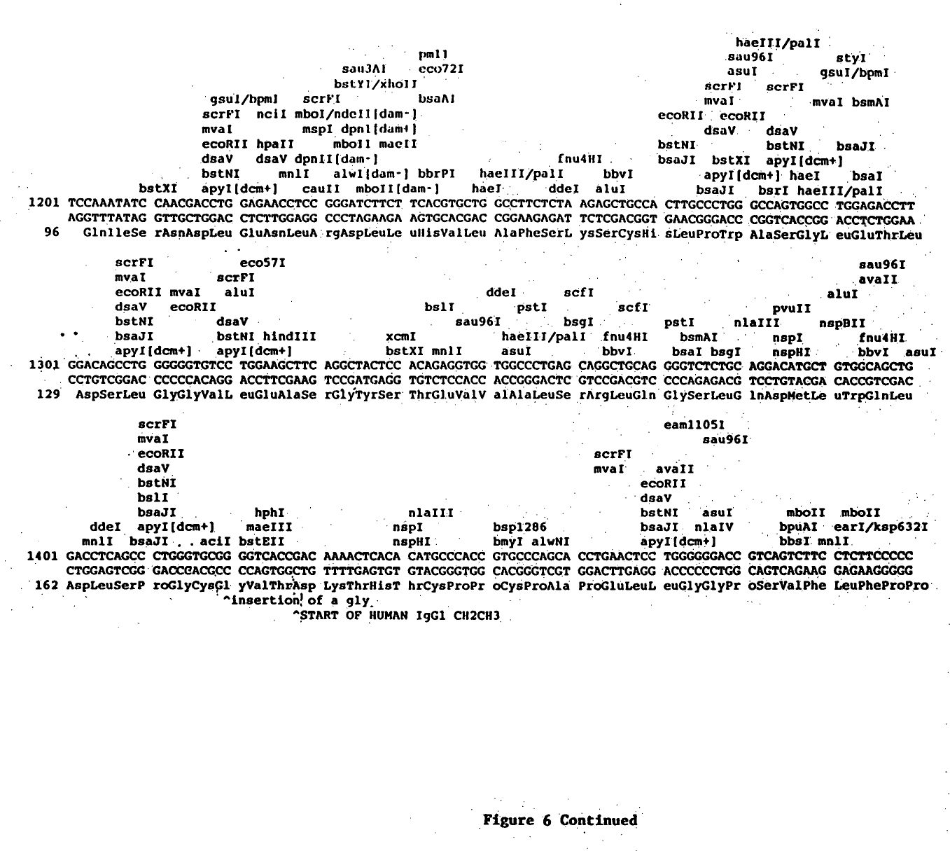

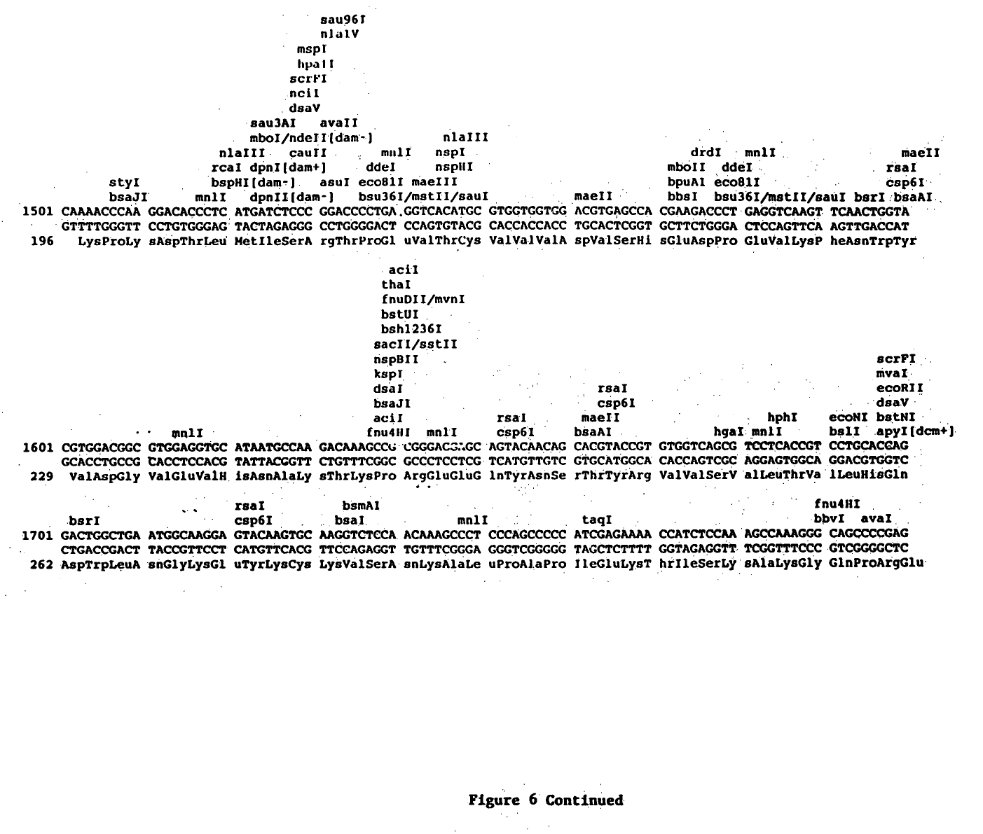

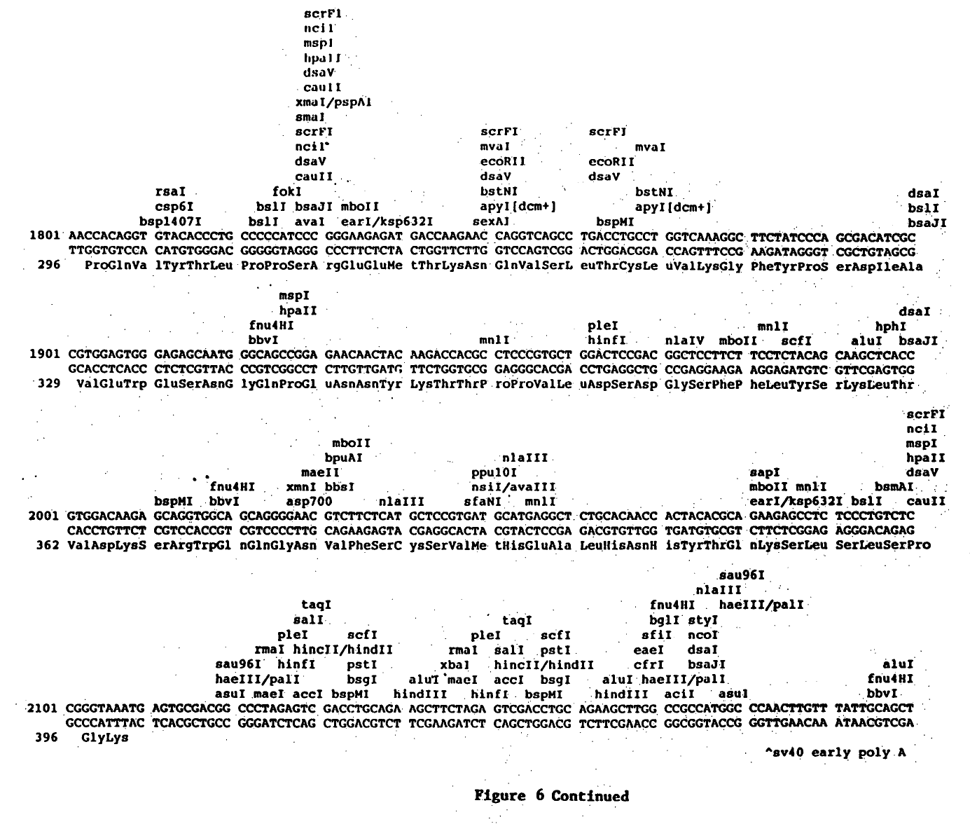

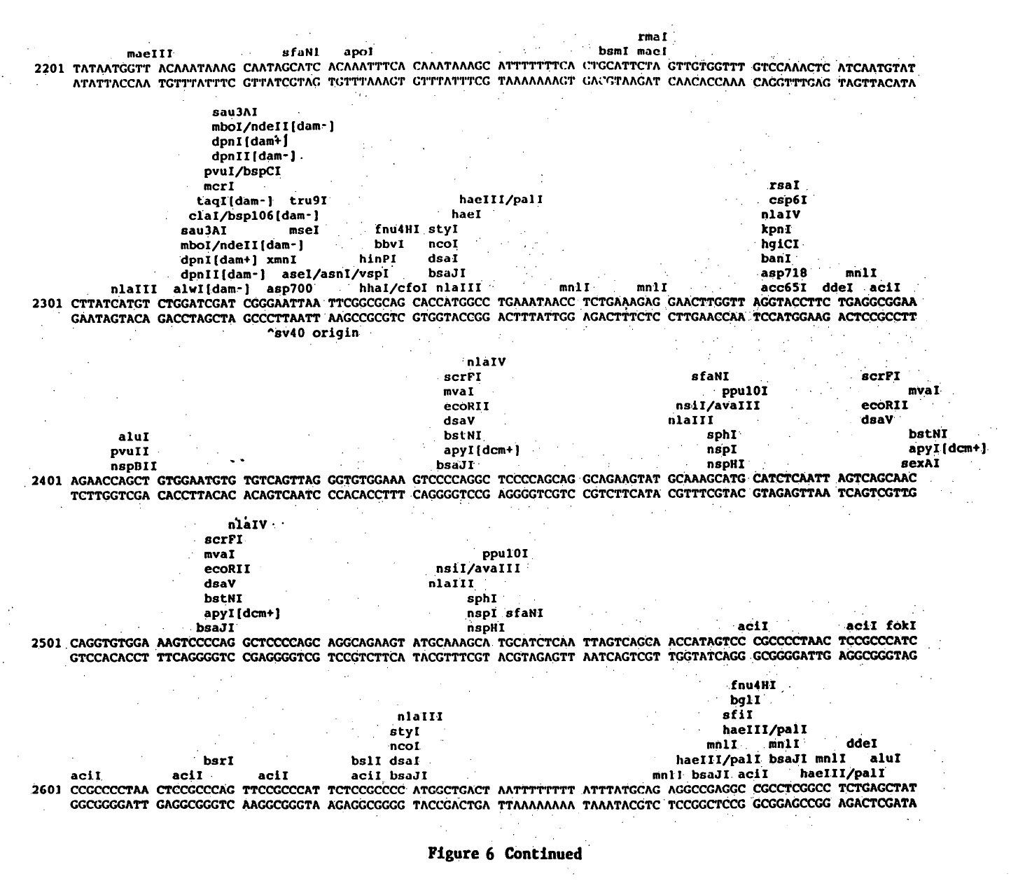

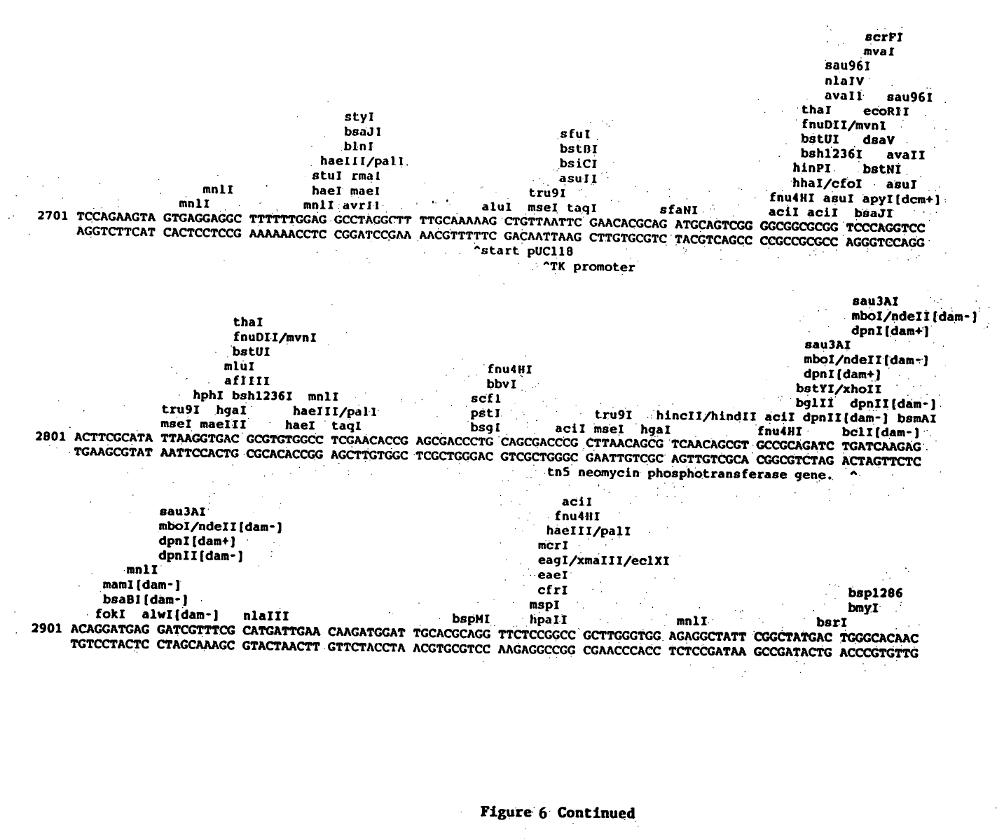

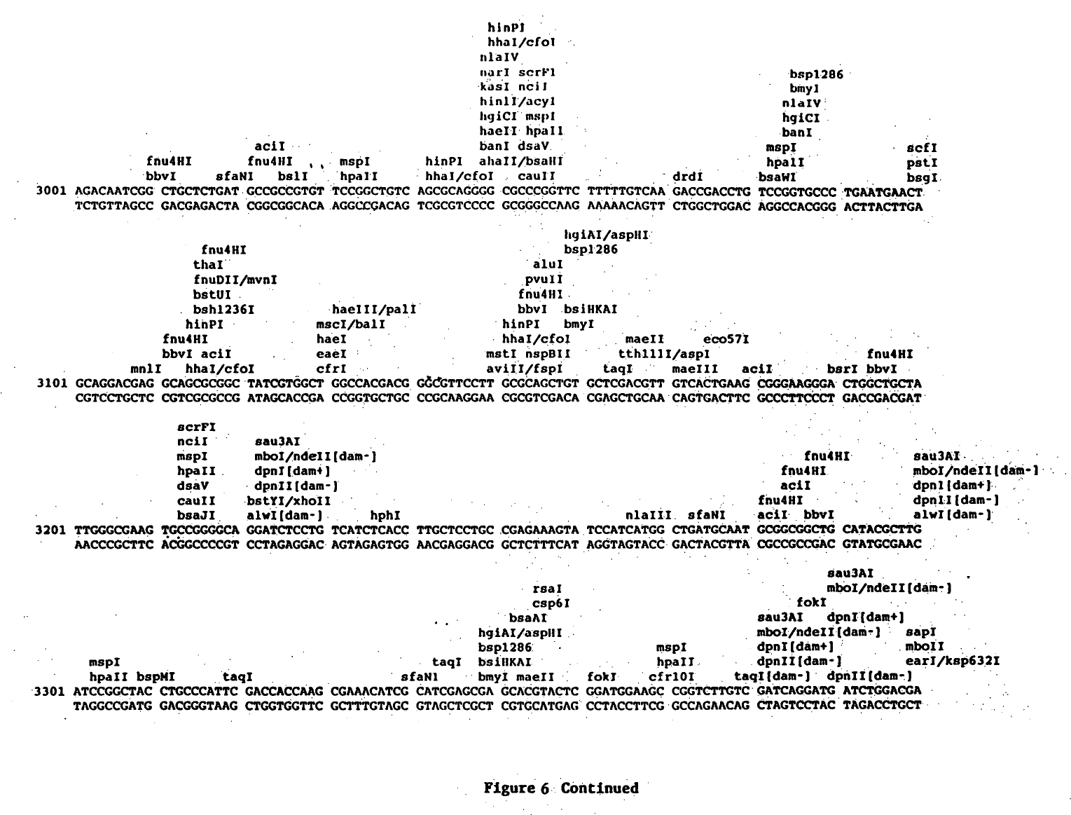

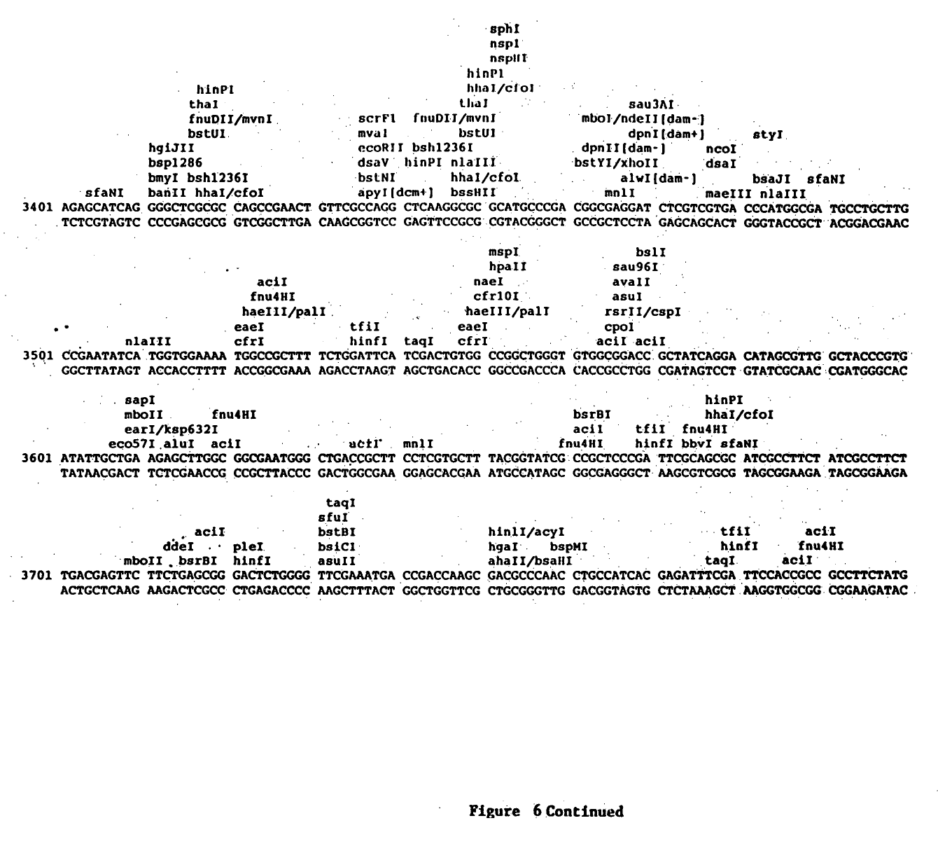

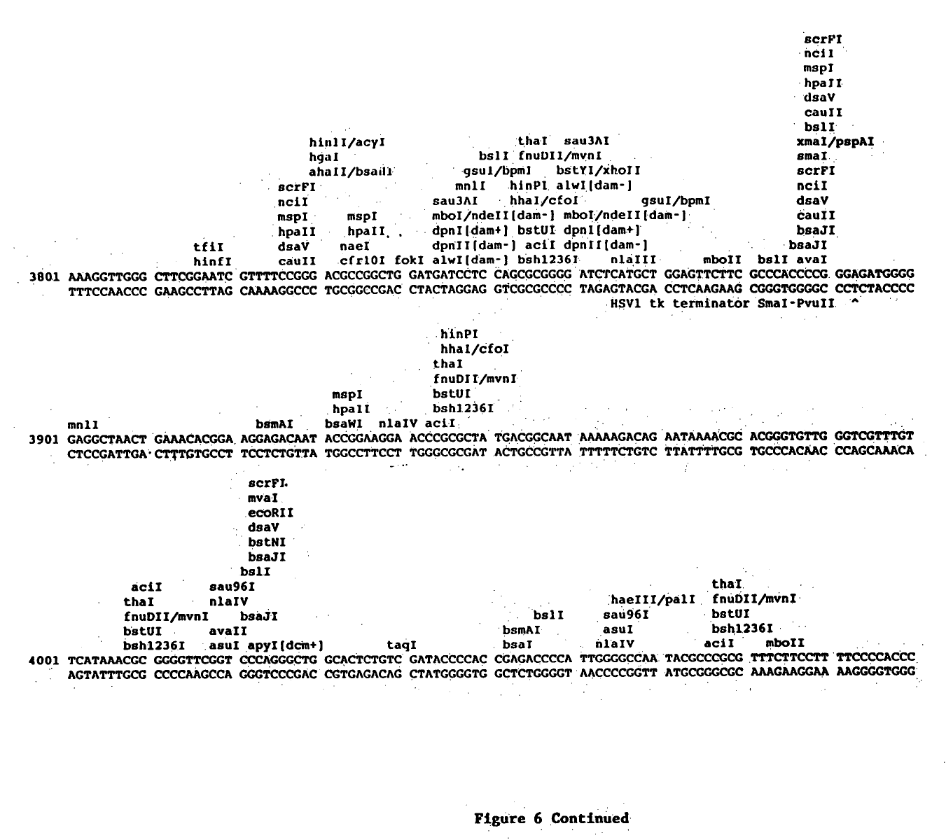

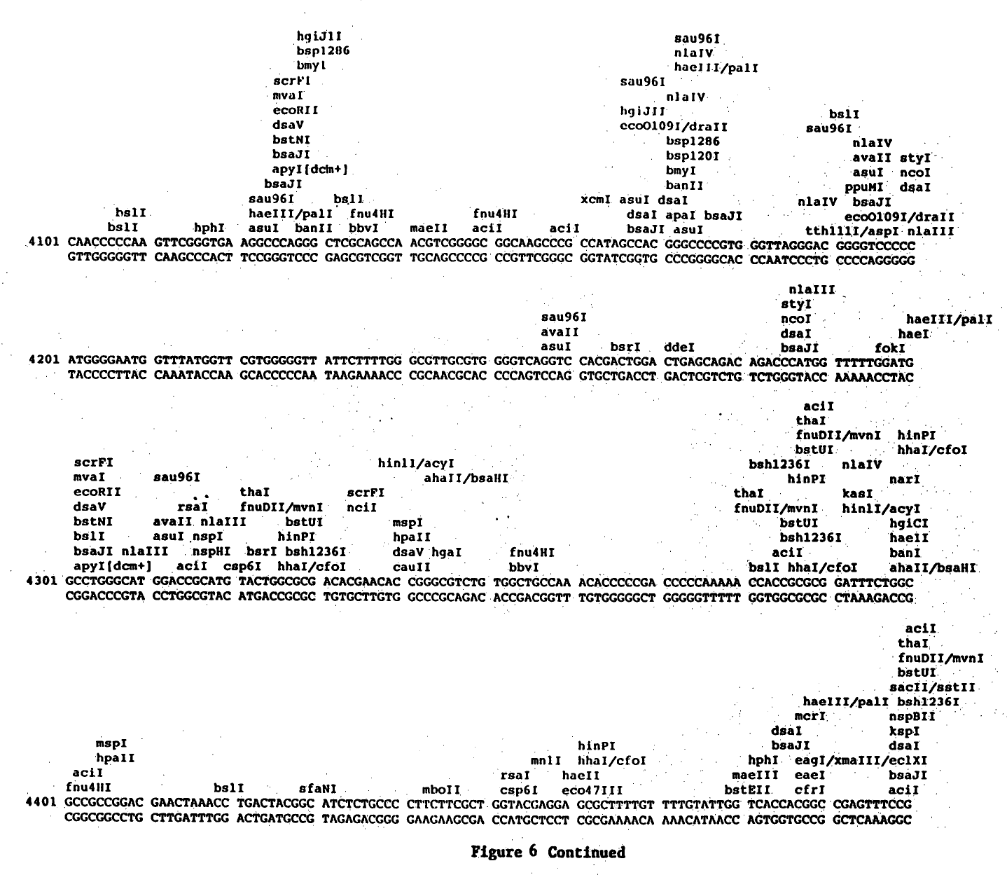

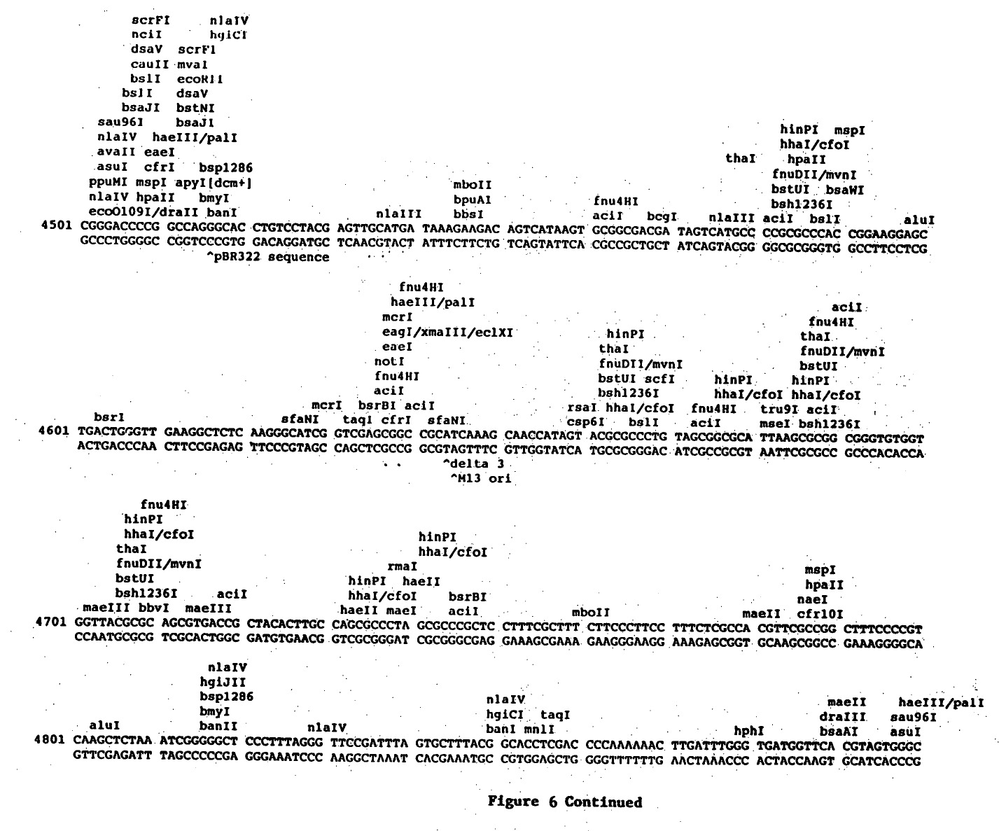

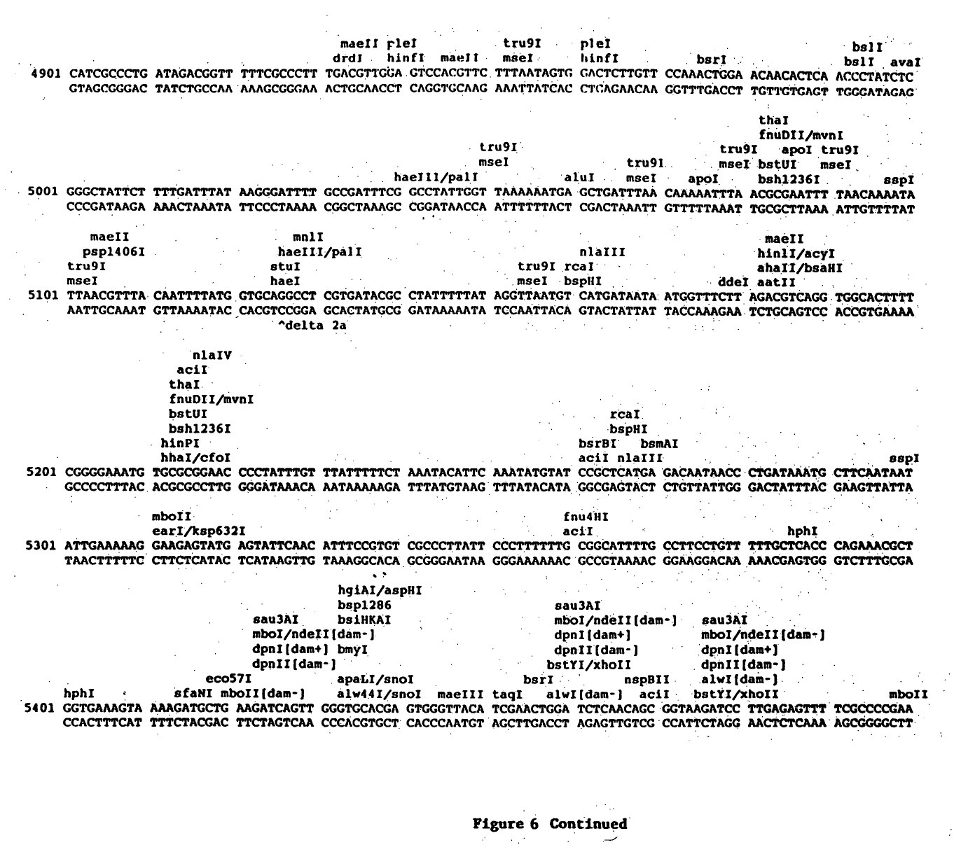

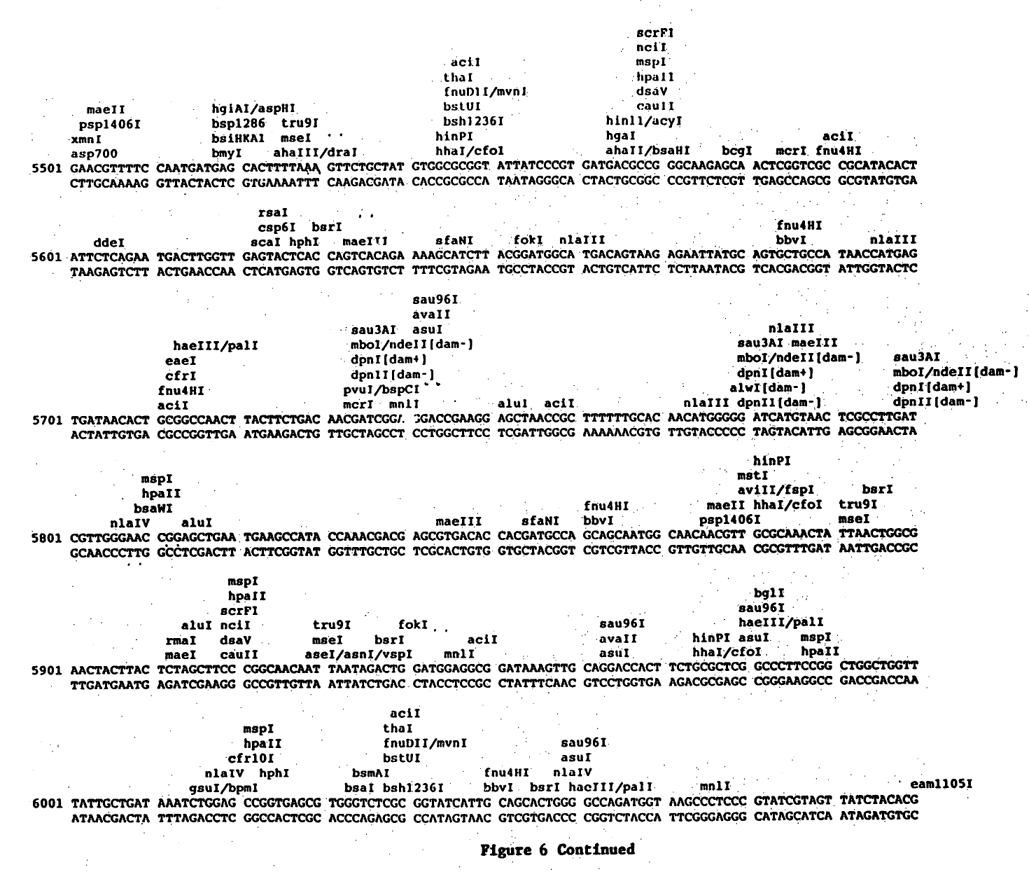

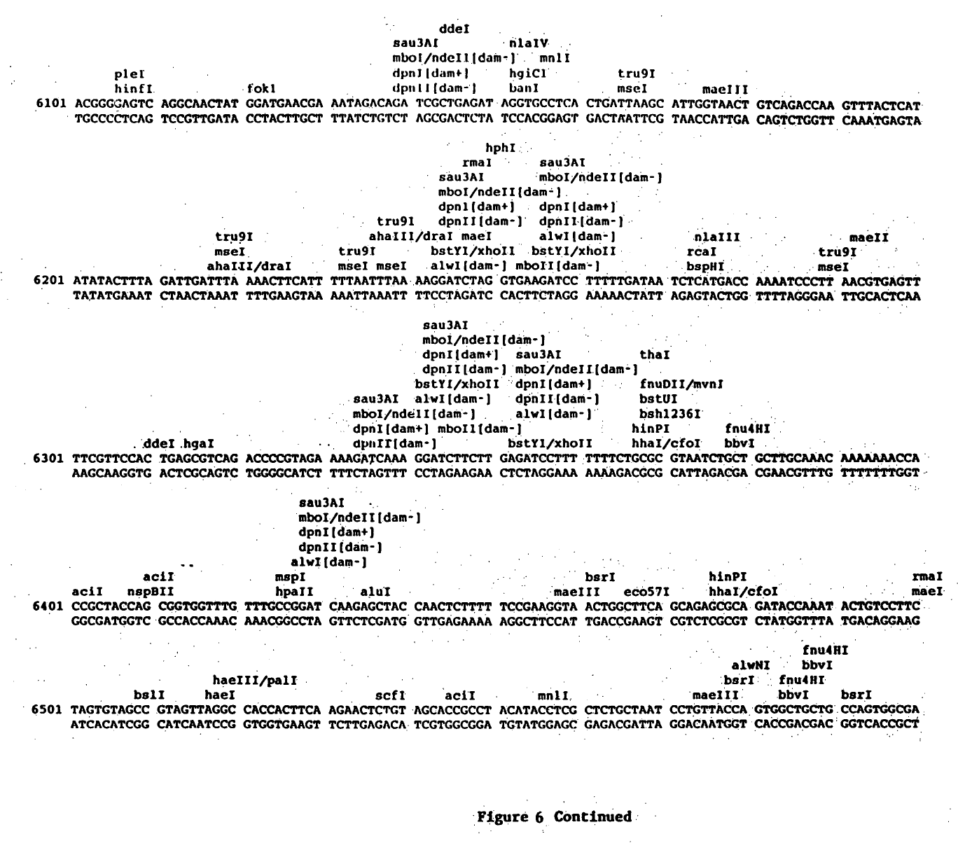

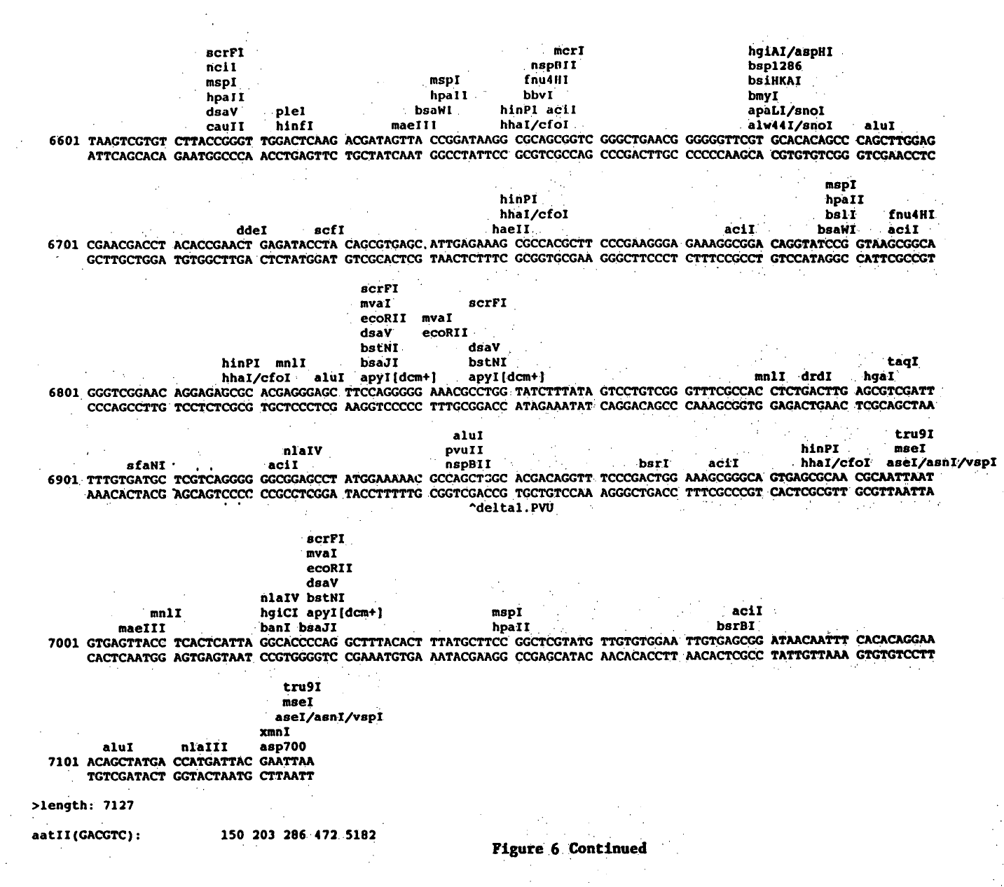

The terms “native” and “native sequence” OB polypeptide are used to refer to an OB polypeptide from any animal species (e.g. human, murine, rabbit, cat, cow, sheep, chicken, porcine, equine, etc.), as occurring in nature, including naturally-occurring alleles, deletion, substitution and/or insertion variants, as currently known or as might be identified in the future, provided that they retain the ability to bind to and, preferably, signal through the OB receptor. Thus, a native human OB polypeptide includes the amino acid sequence between the N-terminus and the cysteine (Cys) at position 167 of the amino acid sequence shown in FIG. 6 (see also SEQ. ID. NO: 2 and FIG. 6 of Zhang et al., supra), and naturally occurring variants of this protein, as currently known or might be identified in the future. Similarly, a “native” or “native sequence” murine OB polypeptide has the amino acid sequence shown in FIG. 6 of Zhang et al., supra, and naturally occurring variants of that polypeptide, as currently known or might be identified in the future. The definition specifically includes variants with or without a glutamine at amino acid position 49, using the amino acid numbering of Zhang et al., supra. The terms “native” and “native sequence” OB polypeptide include the native proteins with or without the initiating N-terminal methionine (Met), and with or without the native signal sequence, either in monomeric or in dimeric form. The native human and murine OB polypeptides known in the art are 167 amino acids long, contain two conserved cysteines, and have the features of a secreted protein. The polypeptide is largely hydrophilic, and the predicted signal sequence cleavage site is at position 21, using the amino acid numbering of Zhang et al., supra. The overall sequence homology of the human and murine sequences is about 84%. The two proteins show a more extensive identity in the N-terminal region of the mature protein, with only four conservative and three non-conservative substitutions among the residues between the signal sequence cleavage site and the conserved Cys at position 117. The molecular weight of OB proteins is about 16 kD in a monomeric form.

A “functional derivative” of a native polypeptide is a compound having a qualitative biological property in common with the native polypeptide. A functional derivative of an OB polypeptide is a compound that has a qualitative biological property in common with a native (human or non-human) OB polypeptide. “Functional derivatives” include but are not limited to, fragments of native polypeptides from any animal species (including humans), and derivatives of native (human and non-human) polypeptides and their fragments, provided that they have a biological activity in common with a corresponding native polypeptide.

“Fragments” comprise regions within the sequence of a mature native OB polypeptide. Preferred fragments of OB polypeptides include the C-terminus of the mature protein, and may contain relatively short deletion(s) at the N-terminus and in other parts of the molecule not required for receptor binding and/or for structural integrity.

The term “derivative” is used to define amino acid sequence variants, and covalent modifications of a native polypeptide, whereas the term “variant” refers to amino acid sequence variants within this definition.

“Biological property” in the context of the definition of “functional derivatives” is defined as either 1) immunological cross-reactivity with at least one epitope of a native polypeptide (e.g. a native OB polypeptide of any species), or 2) the possession of at least one adhesive, regulatory or effector function qualitatively in common with a native polypeptide.

Preferably, the functional derivatives are polypeptides which have at least about 65% amino acid sequence identity, more preferably about 75% amino acid sequence identity, even more preferably at least about 85% amino acid sequence identity, most preferably at least about 95% amino acid sequence identity with a native polypeptide. In the context of the present invention, functional derivatives of native sequence human OB polypeptides preferably show at least 95% amino acid sequence identity with the native OB proteins, and are not immunogenic in the human.

Amino acid sequence identity or homology is defined herein as the percentage of amino acid residues in the candidate sequence that are identical with the residues of a corresponding native polypeptide sequence, after aligning the sequences and introducing gaps, if necessary, to achieve the maximum percent homology, and not considering any conservative substitutions as part of the sequence identity. Neither N- or C-terminal extensions nor insertions shall be construed as reducing identity or homology.

Immunologically cross-reactive as used herein means that the candidate (poly)peptide is capable of competitively inhibiting the qualitative biological activity of a corresponding native polypeptide having this activity with polyclonal antibodies or antisera raised against the known active molecule. Such antibodies and antisera are prepared in conventional fashion by injecting an animal such as a goat or rabbit, for example, subcutaneously with the known native OB protein in complete Freud's adjuvant, followed by booster intraperitoneal or subcutaneous injection in incomplete Freud's.

The term “isolated OB polypeptide” and grammatical variants thereof refer to OB polypeptides (as hereinabove defined) separated from contaminant polypeptides present in the human, other animal species, or in other source from which the polypeptide is isolated.

In general, the term “amino acid sequence variant” refers to molecules with some differences in their amino acid sequences as compared to a reference (e.g. native sequence) polypeptide. The amino acid alterations may be substitutions, insertions, deletions or any desired combinations of such changes in a native amino acid sequence.

Substitutional variants are those that have at least one amino acid residue in a native sequence removed and a different amino acid inserted in its place at the same position. The substitutions may be single, where only one amino acid in the molecule has been substituted, or they may be multiple, where two or more amino acids have been substituted in the same molecule.

Insertional variants are those with one or more amino acids inserted immediately adjacent to an amino acid at a particular position in a native amino acid sequence. Immediately adjacent to an amino acid means connected to either the α-carboxy or α-amino functional group of the amino acid.

Deletional variants are those with one or more amino acids in the native amino acid sequence removed. Ordinarily, deletional variants will have one or two amino acids deleted in a particular region of the molecule.

“Covalent derivatives” include modifications of a native polypeptide or a fragment thereof with an organic proteinaceous or non-proteinaceous derivatizing agent, and post-translational modifications. Covalent modifications are traditionally introduced by reacting targeted amino acid residues with an organic derivatizing agent that is capable of reacting with selected sites or terminal residues, or by harnessing mechanisms of post-translational modifications that function in selected recombinant host cells. Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the OB-immunoglobulin chimeras of the present invention. Other post-translational modifications include hydroxylation of proline and lysine, phosphorylation of hydroxyl groups of seryl, tyrosine or threonyl residues, methylation of the α-amino groups of lysine, arginine, and histidine side chains [T. E. Creighton, Proteins: Structure and Molecular Properties, W. H. Freeman & Co., San Francisco, pp. 79-86 (1983)].

The terms “DNA sequence encoding”, “DNA encoding” and “nucleic acid encoding” refer to the order or sequence of deoxyribonucleotides along a strand of deoxyribonucleic acid. The order of these deoxyribonucleotides determines the order of amino acids along the polypeptide chain. The DNA sequence thus codes for the amino acid sequence.

The terms “replicable expression vector” and “expression vector” refer to a piece of DNA, usually double-stranded, which may have inserted into it a piece of foreign DNA. Foreign DNA is defined as heterologous DNA, which is DNA not naturally found in the host cell. The vector is used to transport the foreign or heterologous DNA into a suitable host cell. Once in the host cell, the vector can replicate independently of the host chromosomal DNA, and several copies of the vector and its inserted (foreign) DNA may be generated. In addition, the vector contains the necessary elements that permit translating the foreign DNA into a polypeptide. Many molecules of the polypeptide encoded by the foreign DNA can thus be rapidly synthesized.

The term “control sequences” refers to DNA sequences necessary for the expression of an operably linked coding sequence in a particular host organism. The control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancer.

Nucleic acid is “operably linked” when it is placed into a functional relationship with another nucleic acid sequence. For example, DNA for a presequence or a secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, then synthetic oligonucleotide adaptors or linkers are used in accord with conventional practice.

In the context of the present invention the expressions “cell”, “cell line”, and “cell culture” are used interchangeably, and all such designations include progeny. Thus, the words “transformants” and “transformed (host) cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

Native immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one and (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-663 (1985); Novotny and Haber, Proc. Natl. Acad. Sci. USA 82, 4592-4596 [1985]).

Depending on the amino acid sequence of the constant region of their heavy chains, immunoglobulins can be assigned to different classes. There are five major classes of immunoglobulins: IgA, IgD, IgE, IgG and IgM, and several of these may be further divided into subclasses (isotypes), e.g. IgG-1, IgG-2, IgG-3, and IgG-4; IgA-1 and IgA-2. The heavy chain constant regions that correspond to the different classes of immunoglobulins are called α, delta, epsilon, γ, and μ, respectively. The subunit structures and three-dimensional configurations of different classes of immunoglobulins are well known. IgA-1 and IgA-2 are monomeric subclasses of IgA, which usually is in the form of dimers or larger polymers. Immunocytes in the gut produce mainly polymeric IgA (also referred to poly-IgA including dimers and higher polymers). Such poly-IgA contains a disulfide-linked polypeptide called the “joining” or “J” chain, and can be transported through the glandular epithelium together with the J-containing polymeric IgM (poly-IgM), comprising five subunits.

Hybridization is preferably performed under “stringent conditions” which means (1) employing low ionic strength and high temperature for washing, for example, 0.015 sodium chloride/0.0015 M sodium citrate/0.1% sodium dodecyl sulfate at 50° C., or (2) employing during hybridization a denaturing agent, such as formamide, for example, 50% (vol/vol) formamide with 0.1% bovine serum albumin/0.1% Ficoll/0.1% polyvinylpyrrolidone/50 nM sodium phosphate buffer at pH 6.5 with 750 mM sodium chloride, 75 mM sodium citrate at 42° C. Another example is use of 50% formamide, 5×SSC (0.75 M NaCl, 0.075 M sodium citrate), 50 mM sodium phosphate (pH 6/8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC and 0.1% SDS.

B. OB Protein-Immunoglobulin Chimeras (Immunoadhesins)

Immunoadhesins are chimeric antibody-like molecules that combine the functional domain(s) of a binding protein (usually a receptor, a cell-adhesion molecule or a ligand) with the an immunoglobulin sequence. The most common example of this type of fusion protein combines the hinge and Fc regions of an immunoglobulin (Ig) with domains of a cell-surface receptor that recognizes a specific ligand. This type of molecule is called an “immunoadhesin”, because it combines “immune” and “adhesion” functions; other frequently used names are “Ig-chimera”, “Ig-” or “Fc-fusion protein”, or “receptor-globulin.”

To date, more than fifty immunoadhesins have been reported in the art. Immunoadhesins reported in the literature include, for example, fusions of the T cell receptor (Gascoigne et al., Proc. Natl. Acad. Sci. USA 84, 2936-2940 [1987]); CD4 (Capon et al., Nature 337, 525-531 [1989]; Traunecker et al., Nature 339, 68-70 [1989]; Zettmeissl et al., DNA Cell Biol. USA9, 347-353 [1990]; Byrn et al., Nature 344, 667-670 [1990]); L-selectin (homing receptor) (Watson et al., J. Cell. Biol. 110, 2221-2229 [1990]; Watson et al., Nature 349,164-167 [1991]); E-selectin [Mulligan et al., J. Immunol. 151, 6410-17 [1993]; Jacob et al., Biochemistry 34, 1210-1217 [1995]); P-selectin (Mulligan et al., supra; Hollenbaugh et al., Biochemistry 34, 5678-84 [1995]); ICAM-1 (Stauton et al., J. Exp. Med. 176, 1471-1476 [1992]; Martin et al., J. Virol. 67, 3561-68 [1993]; Roep et al., Lancet 343, 1590-93 [1994]); ICAM-2 (Damle et al., J. Immunol. 148, 665-71 [1992]); ICAM-3 (Holness et al., J. Biol. Chem. 270, 877-84 [1995]); LFA-3 (Kanner et al., J. Immunol. 148, 2-23-29 [1992]); L1 glycoprotein (Doherty et al., Neuron 14, 57-66 [1995]); TNF-R1 (Ashkenazi et al., Proc. Natl. Acad. Sci. USA 88, 10535-539 [1991]; Lesslauer et al., Eur. J. Immunol. 21, 2883-86 [1991]; Peppel et al., J. Exp. Med. 174, 1483-1489 [1991]); TNF-R2 (Zack et al., Proc. Natl. Acad. Sci. USA 90, 2335-39 [1993]; Wooley et al., J. Immunol. 151, 6602-07 [1993]); CD44 [Aruffo et al., Cell 61, 1303-1313 (1990)]; CD28 and B7 [Linsley et al., J. Exp. Med. 173, 721-730 (1991)]; CTLA-4 [Lisley et al., J. Exp. Med. 174, 561-569 (1991)]; CD22 [Stamenkovic et al., Cell 66.1133-1144 (1991)]; NP receptors [Bennett et al., J. Biol. Chem. 266, 23060-23067 (1991)]; IgE receptor a [Ridgway and Gorman, J. Cell. Biol. 115, abstr. 1448 (1991)]; HGF receptor [Mark, M. R. et al., 1992, J. Biol. Chem. submitted]; IFN-γR α- and β-chain [Marsters et al., Proc. Natl. Acad. Sci. USA 92, 5401-05 [1995]); trk-A, -B, and -C (Shelton et al., J. Neurosci. 15, 477-91 [1995]); IL-2 (Landolfi, J. Immunol. 146, 915-19 [1991]); IL-10 (Zheng et al., J. Immunol. 154, 5590-5600 [1995]).

The simplest and most straightforward immunoadhesin design combines the binding region(s) of the ‘adhesin’ protein with the hinge and Fc regions of an immunoglobulin heavy chain. Ordinarily, when preparing the OB-immunoglobulin chimeras of the present invention, nucleic acid encoding the desired OB polypeptide will be fused C-terminally to nucleic acid encoding the N-terminus of an immunoglobulin constant domain sequence, however N-terminal fusions are also possible. Typically, in such fusions the encoded chimeric polypeptide will retain at least functionally active hinge, CH2 and CH3 domains of the constant region of an immunoglobulin heavy chain. Fusions are also made to the C-terminus of the Fc portion of a constant domain, or immediately N-terminal to the CH1 of the heavy chain or the corresponding region of the light chain. The precise site at which the fusion is made is not critical; particular sites are well known and may be selected in order to optimize the biological activity, secretion or binding characteristics of the OB-immunoglobulin chimeras.

In a preferred embodiment, the sequence of a native, mature OB polypeptide, is fused to the N-terminus of the C-terminal portion of an antibody (in particular the Fc domain), containing the effector functions of an immunoglobulin, e.g. IgG-1. It is possible to fuse the entire heavy chain constant region to the OB sequence. However, more preferably, a sequence beginning in the hinge region just upstream of the papain cleavage site (which defines IgG Fc chemically; residue 216, taking the first residue of heavy chain constant region to be 114 [Kobet et al., supra], or analogous sites of other immunoglobulins) is used in the fusion. In a particularly preferred embodiment, the OB polypeptide sequence is fused to the hinge region and CH2 and CH3 or CH1, hinge, CH2 and CH3 domains of an IgG-1, IgG-2, or IgG-3 heavy chain. The precise site at which the fusion is made is not critical, and the optimal site can be determined by routine experimentation.

In some embodiments, the OB-immunoglobulin chimeras are assembled as multimers, and particularly as homo-dimers or -tetramers (WO 91/08298). Generally, these assembled immunoglobulins will have known unit structures. A basic four chain structural unit is the form in which IgG, IgD, and IgE exist. A four unit is repeated in the higher molecular weight immunoglobulins; IgM generally exists as a pentamer of basic four units held together by disulfide bonds. IgA globulin, and occasionally IgG globulin, may also exist in multimeric form in serum. In the case of multimer, each four unit may be the same or different.

Various exemplary assembled OB-immunoglobulin chimeras within the scope herein are schematically diagrammed below:

(a) AC_{L}-AC_{L};

(b) AC_{H}-[AC_{H}, AC_{L}-AC_{H}, AC_{L}-V_{H}C_{H}, or V_{L}C_{L}-AC_{H}];

(c) AC_{L}-AC_{H}-[AC_{L}-AC_{H}, AC_{L}-V_{H}C_{H}, V_{L}C_{L}-AC_{H}, or V_{L}C_{L}-V_{H}C_{H}];

(d) AC_{L}-V_{H}C_{H}-[AC_{H}, or AC_{L}-V_{H}C_{H}, or V_{L}C_{L}-AC_{H}];

(e) V_{L}C_{L}-AC_{H}-[AC_{L}-V_{H}C_{H}, or V_{L}C_{L}-AC_{H}]; and

(f) [A-Y]ₙ-[V_{L}C_{L}-V_{H}C_{H}]₂,

wherein

each A represents identical or different OB polypeptide amino acid sequences;

V_{L} is an immunoglobulin light chain variable domain;

V_{H} is an immunoglobulin heavy chain variable domain;

C_{L} is an immunoglobulin light chain constant domain;

C_{H} is an immunoglobulin heavy chain constant domain;

n is an integer greater than 1;

Y designates the residue of a covalent cross-linking agent.

In the interests of brevity, the foregoing structures only show key features; they do not indicate joining (J) or other domains of the immunoglobulins, nor are disulfide bonds shown. However, where such domains are required for binding activity, they shall be constructed as being present in the ordinary locations which they occupy in the immunoglobulin molecules.

Alternatively, the OB amino acid sequences can be inserted between immunoglobulin heavy chain and light chain sequences such that an immunoglobulin comprising a chimeric heavy chain is obtained. In this embodiment, the OB polypeptide sequences are fused to the 3′ end of an immunoglobulin heavy chain in each arm of an immunoglobulin, either between the hinge and the CH2 domain, or between the CH2 and CH3 domains. Similar constructs have been reported by Hoogenboom, H. R. et al., Mol. Immunol. 28, 1027-1037 (1991).

Although the presence of an immunoglobulin light chain is not required in the immunoadhesins of the present invention, an immunoglobulin light chain might be present either covalently associated to an OB protein-immunoglobulin heavy chain fusion polypeptide, or directly fused to the OB polypeptide. In the former case, DNA encoding an immunoglobulin light chain is typically coexpressed with the DNA encoding the OB-immunoglobulin heavy chain fusion protein. Upon secretion, the hybrid heavy chain and the light chain will be covalently associated to provide an immunoglobulin-like structure comprising two disulfide-linked immunoglobulin heavy chain-light chain pairs. Method suitable for the preparation of such structures are, for example, disclosed in U.S. Pat. No. 4,816,567 issued 28 Mar. 1989.

In a preferred embodiment, the immunoglobulin sequences used in the construction of the immunoadhesins of the present invention are from an IgG immunoglobulin heavy chain constant domain. For human immunoadhesins, the use of human IgG-1 and IgG-3 immunoglobulin sequences is preferred. A major advantage of using IgG-1 is that IgG-1 immunoadhesins can be purified efficiently on immobilized protein A. In contrast, purification of IgG-3 requires protein G, a significantly less versatile medium. However, other structural and functional properties of immunoglobulins should be considered when choosing the Ig fusion partner for a particular immunoadhesin construction. For example, the IgG-3 hinge is longer and more flexible, so it can accommodate larger ‘adhesin’ domains that may not fold or function properly when fused to IgG-1. Possible IgG-based immunoadhesin structures are shown in FIG. 3a-c. While IgG immunoadhesins are typically mono- or bivalent, other Ig subtypes like IgA and IgM may give rise to dimeric or pentameric structures, respectively, of the basic Ig homodimer unit. A typical IgM-based multimeric immunoadhesin is illustrated in FIG. 3d. Multimeric immunoadhesins are advantageous in that they can bind their respective targets with greater avidity than their IgG-based counterparts. Reported examples of such structures are CD4-IgM (Traunecker et al., supra); ICAM-IgM (Martin et al., J. Virol. 67, 3561-68 [1993]); and CD2-IgM (Arulanandam et al., J. Exp. Med. 177, 1439-50 [1993]).

For OB-Ig immunoadhesins, which are designed for in vivo application, the pharmacokinetic properties and the effector functions specified by the Fc region are important as well. Although IgG-1, IgG-2 and IgG-4 all have in vivo half-lives of 21 days, their relative potencies at activating the complement system are different. IgG4 does not activate complement, and IgG-2 is significantly weaker at complement activation than IgG-1. Moreover, unlike IgG-1, IgG-2 does not bind to Fc receptors on mononuclear cells or neutrophils. While IgG-3 is optimal for complement activation, its in vivo half-life is approximately one third of the other IgG isotypes. Another important consideration for immunoadhesins designed to be used as human therapeutics is the number of allotypic variants of the particular isotype. In general, IgG isotypes with fewer serologically-defined allotypes are preferred. For example, IgG-1 has only four serologically-defined allotypic sites, two of which (G1m and 2) are located in the Fc region; and one of these sites G1m1, is non-immunogenic. In contrast, there are 12 serologically-defined allotypes in IgG-3, all of which are in the Fc region; only three of these sites (G3 m5, 11 and 21) have one allotype which is nonimmunogenic. Thus, the potential immunogenicity of a γ3 immunoadhesin is greater than that of a γ1 immunoadhesin.

In designing the OB-Ig immunoadhesins of the present invention regions that are not required for receptor binding, the structural integrity (e.g. proper folding) and/or biological activity of the molecule, may be deleted. In such structures, it is important to place the fusion junction at residues that are located between domains, to avoid misfolding. With respect to the parental immunoglobulin, a useful joining point is just upstream of the cysteines of the hinge that form the disulfide bonds between the two heavy chains. In a frequently used design, the codon for the C-terminal residue of the “adhesin” (OB) part of the molecule is placed directly upstream of the codons for the sequence DKTHTCPPCP of the IgG1 hinge region.

OB-Ig immunoadhesins are most conveniently constructed by fusing the cDNA sequence encoding the OB portion in-frame to an Ig cDNA sequence. However, fusion to genomic Ig fragments can also be used (see, e.g. Gascoigne et al., Proc. Natl. Acad. Sci. USA 84, 2936-2940 [1987]; Aruffo et al., Cell 61, 1303-1313 [1990]; Stamenkovic et al., Cell 66, 1133-1144 [1991]). The latter type of fusion requires the presence of Ig regulatory sequences for expression. cDNAs encoding IgG heavy-chain constant regions can be isolated based on published sequence from cDNA libraries derived from spleen or peripheral blood lymphocytes, by hybridization or by polymerase chain reaction (PCR) techniques. Murine OB cDNA can, for example, be obtained by PCR from a mouse adipose tissue cDNA library (Clontech), using primers designed based on the sequence of Zhang et al. Human OB cDNA can be obtained in a similar manner. Alternatively, the mouse OB gene can be used as a probe to isolate human adipose tissue cDNA clones (Clontech), e.g. from a λgtII library, as described by Zhang et al. The cDNAs encoding the ‘adhesin’ and the Ig parts of the immunoadhesin are inserted in tandem into a plasmid vector that directs efficient expression in the chosen host cells. For expression in mammalian cells pRK5-based vectors (Schall et al., Cell 61, 361-370 [1990]), pRK7-vectors and CDM8-based vectors (Seed, Nature 329, 840 [1989]) are preferred. (pRK7 is identical to pRK5 except that the order of the endonuclease restriction sites in the polylinker region between ClaI and HindIII is reversed. See U.S. Pat. No. 5,108,901 issued 28 Apr. 1992.). The exact junction can be created by removing the extra sequences between the designed junction codons using oligonucleotide-directed deletional mutagenesis (Zoller and Smith, Nucleic Acids Res. 10, 6487 [1982]; Capon et al., Nature 337, 525-531 [1989]). Synthetic oligonucleotides can be used, in which each half is complementary to the sequence on either side of the desired junction; ideally, these are 36 to 48-mers. Alternatively, PCR technique can be used to join the two parts of the molecule in-frame with an appropriate vector.

Immunoadhesins can be expressed efficiently in a variety of host cells, including myeloma cell lines, Chinese Hamster ovary (CHO) cells, monkey COS cells, human embryonic kidney 293 cells, and baculovirus infected insect cells. In these systems, the immunoadhesin polypeptides are assembled and secreted into the cell culture medium. Yeasts, e.g. Saccharomyces cerevisiae, Pichia pastoris, etc., and bacterial cells, preferably E. coli, can also be used as hosts. The OB-immunoglobulin chimeras can be expressed in yeast, for example, similarly to the process described for the expression of the OB proteins by Leiber et al., Crit. Res. Food Sci. Nutr. 33, 351 (1993); Friedman and Leibel, Cell 69, 217 (1992); and Beavis and Chait, Proc. Natl. Acad. Sci. USA 87, 6873 (1990). Thus, the coding sequences can be subcloned into a yeast plasmid, such as the yeast expression plasmid pPIC.9 (Invitrogen). This vector directs secretion of heterologous proteins from the yeast into the culture media. According to Halaas et al., supra, expression of mouse and human OB genes in Saccharomyces cerevisiae transformed with this vector yields a secreted 16-kD protein, which is an unprocessed OB protein lacking the signal sequence. Expression of the mouse or human OB-immunoglobulin chimeras in E. coli can, for example, be performed on the analogy of the procedure described by Halaas et al., supra. The coding sequences of mouse and human OB-immunoglobulin chimeras can be subcloned into the PET15b expression vector (Novagen) and expressed in E. coli (BL21 (DE3)pIYsS) through use of the T7 E. coli RNA polymerase system. Alternatively, the fusion protein can be expressed in E. coli by inserting the coding sequence in frame with the secretion sequence of the E. coli heat stable enterotoxin II, downstream of the E. coli alkaline phosphatase promoter (Chang et al., Gene 55, 189-96 [1987]).

The choice of host cell line for the expression of OB-Ig immunoadhesins depends mainly on the expression vector. Another consideration is the amount of protein that is required. Milligram quantities often can be produced by transient transfections. For example, the adenovirus EIA-transformed 293 human embryonic kidney cell line can be transfected transiently with pRK5- and pRK7-based vectors by a modification of the calcium phosphate method to allow efficient immunoadhesin expression. This method is illustrated in the examples. CDM8-based vectors can be used to transfect COS cells by the DEAE-dextran method (Aruffo et al., Cell 61, 1303-1313 (1990); Zettmeissl et al., DNA Cell Biol. (US) 9, 347-353 (1990)]. If larger amounts of protein are desired, the immunoadhesin can be expressed after stable transfection of a host cell line. For example, a pRK5- or pRK7-based vector can be introduced into Chinese hamster ovary (CHO) cells in the presence of an additional plasmid encoding dihydrofolate reductase (DHFR) and conferring resistance to G418. Clones resistant to G418 can be selected in culture; these clones are grown in the presence of increasing levels of DHFR inhibitor methotrexate; clones are selected, in which the number of gene copies encoding the DHFR and immunoadhesin sequences is co-amplified. If the immunoadhesin contains a hydrophobic leader sequence at its N-terminus, it is likely to be processed and secreted by the transfected cells. The expression of immunoadhesins with more complex structures may require uniquely suited host cells; for example, components such as light chain or J chain may be provided by certain myeloma or hybridoma cell hosts [Gascoigne et al., 1987, supra; Martin et al., J. Virol. 67, 3561-3568 (1993)].

The expression of immunoadhesins with more complex oligomeric structures may require uniquely suited host cells; for example, components such as light chain or J chain may be provided by certain myeloma or hybridoma cell hosts (Gascoigne et al., supra; Martin et al., J. Immunol. 67, 3561-68 [1993]).

Immunoadhesins can be conveniently purified by affinity chromatography. The suitability of protein A as an affinity ligand depends on the species and isotype of the immunoglobulin Fc domain that is used in the chimera. Protein A can be used to purify immunoadhesins that are based on human γ1, γ2, or γ4 heavy chains [Lindmark et al., J. Immunol. Meth. 62, 1-13 (1983)]. Protein G is recommended for all mouse isotypes and for human γ3 [Guss et al., EMBO J. 5, 15671575 (1986)]. The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. The conditions for binding an immunoadhesin to the protein A or G affinity column are dictated entirely by the characteristics of the Fc domain; that is, its species and isotype. Generally, when the proper ligand is chosen, efficient binding occurs directly from unconditioned culture fluid. One distinguishing feature of immunoadhesins is that, for human γ1 molecules, the binding capacity for protein A is somewhat diminished relative to an antibody of the same Fc type. Bound immunoadhesin can be efficiently eluted either at acidic pH (at or above 3.0), or in a neutral pH buffer containing a mildly chaotropic salt. This affinity chromatography step can result in an immunoadhesin preparation that is >95% pure.

Other methods known in the art can be used in place of, or in addition to, affinity chromatography on protein A or G to purify immunoadhesins. Immunoadhesins behave similarly to antibodies in thiophilic gel chromatography [Hutchens and Porath, Anal. Biochem. 159,217-226 (1986)] and immobilized metal chelate chromatography [Al-Mashikhi and Makai, J. Dairy Sci. 71, 1756-1763 (1988)]. In contrast to antibodies, however, their behavior on ion exchange columns is dictated not only by their isoelectric points, but also by a charge dipole that may exist in the molecules due to their chimeric nature. Microheterogeneity of charge can also be a factor for immunoadhesins in which the adhesin portion of the molecule is glycosylated and contains sialic acid. A specific purification protocol is described in the examples.

Results with the numerous immunoadhesins produced so far show that the fusion of the adhesin portion to an Fc region usually does not perturb the folding of the individual domains. Both the adhesin and the immunoglobulin regions appear to fold correctly, and the Fc portion retins many of the effector functions that are characteristic of antibodies, such as binding to Fc receptors.

Methods generally applicable for the construction, expression and purification of immunoadhesins are described, for example, in U.S. Pat. No. 5,225,538 (issued 6 Jul. 1993) and U.S. Pat. No. 5,455,165 (issued 30 Oct. 1995), the disclosures of which are hereby expressly incorporated by reference. Immunoadhesin construction, expression, purification and various immunoadhesins designs are also described in the review articles by Ashkenazi and Chamow, Methods in Enzymology 8, 104-115 (1995), and Peach and Linsley, Methods in Enzymology 8, 116-123 (1995), the disclosures of which, along with the references cited therein, is hereby expressly incorporated by reference.

C. Other Long Half-Life OB Derivatives

Other derivatives of the OB proteins, which possess a longer half-life than the native molecules comprise the OB protein or an OB-immunoglobulin chimera, covalently bonded to a nonproteinaceous polymer. The nonproteinaceous polymer ordinarily is a hydrophilic synthetic polymer, i.e., a polymer not otherwise found in nature. However, polymers which exist in nature and are produced by recombinant or in vitro methods are useful, as are polymers which are isolated from native sources. Hydrophilic polyvinyl polymers fall within the scope of this invention, e.g. polyvinylalcohol and polyvinylpyrrolidone. Particularly useful are polyalkylene ethers such as polyethylene glycol (PEG); polyelkylenes such as polyoxyethylene, polyoxypropylene, and block copolymers of polyoxyethylene and polyoxypropylene (Pluronics); polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparon. The polymer prior to cross-linking need not be, but preferably is, water soluble, but the final conjugate must be water soluble. In addition, the polymer should not be highly immunogenic in the conjugate form, nor should it possess viscosity that is incompatible with intravenous infusion or injection if it is intended to be administered by such routes.

Preferably the polymer contains only a single group which is reactive. This helps to avoid cross-linking of protein molecules. However, it is within the scope herein to optimize reaction conditions to reduce cross-linking, or to purify the reaction products through gel filtration or chromatographic sieves to recover substantially homogenous derivatives.

The molecular weight of the polymer may desirably range from about 100 to 500,000, and preferably is from about 1,000 to 20,000. The molecular weight chosen will depend upon the nature of the polymer and the degree of substitution. In general, the greater the hydrophilicity of the polymer and the greater the degree of substitution, the lower the molecular weight that can be employed. Optimal molecular weights will be determined by routine experimentation.

The polymer generally is covalently linked to the OB-protein or to the OB-immunoglobulin chimeras though a multifunctional crosslinking agent which reacts with the polymer and one or more amino acid or sugar residues of the OB protein or OB-immunoglobulin chimera to be linked. However, it is within the scope of the invention to directly crosslink the polymer by reacting a derivatized polymer with the hybrid, or via versa.

The covalent crosslinking site on the OB protein or OB-Ig includes the N-terminal amino group and epsilon amino groups found on lysine residues, as well as other amino, imino, carboxyl, sulfhydryl, hydroxyl or other hydrophilic groups. The polymer may be covalently bonded directly to the hybrid without the use of a multifunctional (ordinarily bifunctional) crosslinking agent. Covalent binding to amino groups is accomplished by known chemistries based upon cyanuric chloride, carbonyl diimidazole, aldehyde reactive groups (PEG alkoxide plus diethyl acetal of bromoacetaldehyde; PEG plus DMSO and acetic anhydride, or PEG chloride plus the phenoxide of 4-hydroxybenzaldehyde, succinimidyl active esters, activated dithiocarbonate PEG, 2,4,5-trichlorophenylcloroformate or P-nitrophenylcloroformate activated PEG.) Carboxyl groups are derivatized by coupling PEG-amine using carbodiimide.

Polymers are conjugated to oligosaccharide groups by oxidation using chemicals, e.g. metaperiodate, or enzymes, e.g. glucose or galactose oxidase, (either of which produces the aldehyde derivative of the carbohydrate), followed by reaction with hydrazide or amino derivatized polymers, in the same fashion as is described by Heitzmann et al., P.N.A.S., 71, 3537-41 (1974) or Bayer et al., Methods in Emzymology 62, 310 (1979), for the labeling of oligosaccharides with biotin or avidin. Further, other chemical or enzymatic methods which have been used heretofore to link oligosaccharides are particularly advantageous because, in general, there are fewer substitutions than amino acid sites for derivatization, and the oligosaccharide products thus will be more homogenous. The oligosaccharide substituents also are optionally modified by enzyme digestion to remove sugars, e.g. by neuraminidase digestion, prior to polymer derivatization.

The polymer will bear a group which is directly reactive with an amino acid side chain, or the N- or C-terminus of the polypeptide linked, or which is reactive with the multifunctional cross-linking agent. In general, polymers bearing such reactive groups are known for the preparation of immobilized proteins. In order to use such chemistries here, one should employ a water soluble polymer otherwise derivatized in the same fashion as insoluble polymers heretofore employed for protein immobilization. Cyanogen bromide activation is a particularly useful procedure to employ in crosslinking polysaccharides.

“Water soluble” in reference to the starting polymer means that the polymer or its reactive intermediate used for conjugation is sufficiently water soluble to participate in a derivatization reaction.

“Water soluble” in reference to the polymer conjugate means that the conjugate is soluble in physiological fluids such as blood.

The degree of substitution with such a polymer will vary depending upon the number of reactive sites on the protein, whether all or a fragment of the protein is used, whether the protein is a fusion with a heterologous protein (e.g. an OB-immunoglobulin chimera), the molecular weight, hydrophilicity and other characteristics of the polymer, and the particular protein derivatization sites chosen. In general, the conjugate contains about from 1 to 10 polymer molecules, while any heterologous sequence may be substituted with an essentially unlimited number of polymer molecules so long as the desired activity is not significantly adversely affected. The optimal degree of cross-linking is easily determined by an experimental matrix in which the time, temperature and other reaction conditions are varied to change the degree of substitution, after which the ability of the conjugates to function in the desired fashion is determined.

The polymer, e.g. PEG, is cross-linked by a wide variety of methods known per se for the covalent modification of proteins with nonproteinaceous polymers such as PEG. Certain of these methods, however, are not preferred for the purposes herein. Cyanuronic chloride chemistry leads to many side reactions, including protein cross-linking. In addition, it may be particularly likely to lead to inactivation of proteins containing sulfhydryl groups. Carbonyl diimidazole chemistry (Beauchamp et al., Anal Biochem. 131, 25-33 [1983]) requires high pH (>8.5), which can inactivate proteins. Moreover, since the “activated PEG” intermediate can react with water, a very large molar excess of “activated PEG” over protein is required. The high concentrations of PEG required for the carbonyl diimidazole chemistry also led to problems in purification, as both gel filtration chromatography and hydrophilic interaction chromatography are adversely affected. In addition, the high concentrations of “activated PEG” may precipitate protein, a problem that per se has been noted previously (Davis, U.S. Pat. No. 4,179,337). On the other hand, aldehyde chemistry (Royer, U.S. Pat. No. 4,002,531) is more efficient since it requires only a 40-fold molar excess of PEG and a 1-2 hr incubation. However, the manganese dioxide suggested by Royer for preparation of the PEG aldehyde is problematic “because of the pronounced tendency of PEG to form complexes with metal-based oxidizing agents” (Harris et al., J. Polym. Sci. Polyn. Chem. Ed. 22, 341-52 [1984]). The use of a Moffatt oxidation, utilizing DMSO and acetic anhydride, obviates this problem. In addition, the sodium borohydride suggested by Royer must be used at high pH and has a significant tendency to reduce disulfide bonds. In contrast, sodium cyanoborohydride, which is effective at neutral pH and has very little tendency to reduce disulfide bonds is preferred.

Functionalized PEG polymers to modify the OB protein or OB-Ig chimeras of the present invention are available from Shearwater Polymers, Inc. (Huntsville, Ala.). Such commercially available PEG derivatives include, but are not limited to, amino-PEG, PEG amino acid esters, PEG-hydrazide, PEG-thiol, PEG-succinate, carboxymethylated PEG, PEG-propionic acid, PEG amino acids, PEG succinimidyl succinate, PEG succinimidyl propionate, succinimidyl ester of carboxymethylated PEG, succinimidyl carbonate of PEG, succinimidyl esters of amino acid PEGs, PEG-oxycarbonylimidazole, PEG-nitrophenyl carbonate, PEG tresylate, PEG-glycidyl ether, PEG-aldehyde, PEG vinylsulfone, PEG-maleimide, PEG-orthopyridyl-disulfide, heterofunctional PEGs, PEG vinyl derivatives, PEG silanes, and PEG phospholides. The reaction conditions for coupling these PEG derivatives will vary depending on the protein, the desired degree of PEGylation, and the PEG derivative utilized. Some factors involved in the choice of PEG derivatives include: the desired point of attachment (lysine or cysteine), hydrolytic stability and reactivity of the derivatives, stability, toxicity and antigenicity of the linkeage, suitability for analysis, etc. Specific instructions for the use of any particular derivative are available from the manufacturer.

The long half-life conjugates of this invention are separated from the unreacted starting materials by gel filtration. Heterologous species of the conjugates are purified from one another in the same fashion. The polymer also may be water-insoluble, as a hydrophilic gel.

The conjugates may also be purified by ion-exchange chromatography. The chemistry of many of the electrophilically activated PEG's results in a reduction of amino group charge of the PEGylated product. Thus, high resolution ion exchange chromatography can be used to separate the free and conjugated proteins, and to resolve species with different levels of PEGylation. In fact, the resolution of different species (e.g. containing one or two PEG residues) is also possible due to the difference in the ionic properties of the unreacted amino acids.

D. The Use of the OB-Immunoglobulin Chimeras and Other Long Half-Life Derivatives

The OB-immunoglobulin chimeras and other long half-life OB derivatives of the present invention are useful for weight reduction, and specifically, in the treatment of obesity and other disorders associated with the abnormal expression or function of the OB gene. Our studies indicate that the OB-immunoglobulin chimeras and other long half-life OB derivatives, e.g. PEGylated OB, reduce the food intake and increase the energy use of animals treated, and are therefore very effective in reducing the weight of both obese and normal subjects. For testing purposes, the molecules of the present invention may be dissolved in phosphate-buffered saline (PBS) (pH 7.4), and administered by intravenous or subcutaneous injection, or infusion.

The long acting OB-derivatives of the present invention may further be used to treat other metabolic disorders such as diabetes and bulimia. The OB protein has been shown to reduce insulin levels in animals, and could be useful to reduce excessive levels of insulin in human patients. The reduction of insulin levels in obese or non-obese patients (e.g. Type I or II diabetics) could restore or improve the insulin-sensitivity of such patients.

In addition, the long half-life OB-derivatives can be used for the treatment of kidney ailments, hypertension, and lung disfunctions, such as emphysema. The OB protein might also cause a mitogenic response in receptor-bearing tissues, acting as a growth factor for these cells.

Therapeutic formulations of the present invention are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or PEG.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes, prior to or following lyophilization and reconstitution.

Therapeutic compositions herein generally are placed into a container having a sterile access port, for example, an intravenous solution bag or vial having a stopper pierceable by a hypodermic injection needle.

The route of administration is in accord with known methods, e.g. injection or infusion by intravenous, intraperitoneal, etc. routes. Sustained released formulations are also foreseen. Suitable examples of sustained release preparations include semipermeable polymer matrices in the form of shaped articles, e.g. films, or microcapsules. Sustained release matrices include polyesters, hydrogels, polylactides (U.S. Pat. No. 3,773,919, EP 58,481), copolymers of L-glutamic acid and gamma ethyl-L-glutamate (U. Sidman et al., 1983, “Biopolymers” 22 (1): 547-556), poly(2-hydroxyethyl-methacrylate) (R. Langer, et al., 1981, “J. Biomed. Mater. Res.” 15: 167-277 and R. Langer, 1982, Chem. Tech.” 12:98-105), ethylene vinyl acetate (R. Langer et al., Id.) or poly-D-(−)-3-hydroxybutyric acid (EP 133,988A). Sustained release compositions also include liposomes. Liposomes containing a molecule within the scope of the present invention are prepared by methods known per se: DE3,218,121A; Epstein et al., 1985, “Proc. Natl. Acad. Sci. USA” 82: 3688-3692; Hwang et al., 1980, “Proc. Natl. Acad. Sci. USA” 77: 4030-4034; EP 52322A; EP 36676A; EP 88046A; EP 143949A; EP 142641A; Japanese patent application 83-118008; U.S. Pat. Nos. 4,485,045 and 4,544,545; and EP 102,324A. Ordinarily the liposomes are of the small (about 200-800 Angstroms) unilamelar type in which the lipid content is greater than about 30 mol. % cholesterol, the selected proportion being adjusted for the optimal therapy.

An effective amount of a molecule of the present invention to be employed therapeutically will depend, for example, upon the therapeutic objectives, the route of administration, and the condition of the patient. Accordingly, it will be necessary for the therapist to titer the dosage and modify the route of administration as required to obtain the optimal therapeutic effect. A typical daily dosage might range from about 1 μg/kg to up to 100 mg/kg or more, depending on the factors mentioned above. Typically, the clinician will administer a molecule of the present invention until a dosage is reached that provides the required biological effect. The progress of this therapy is easily monitored by conventional assay techniques. If the purpose of the treatment is weight reduction, the therapy is normally continued until a desired body weight is reached.

Non-therapeutic uses of the OB protein-immunoglobulin fusions of the present invention include their use to identify and purify OB receptors. The identification and expression cloning of an OB receptor, using an OB protein-immunoadhesin is described in a Reference Example hereinbelow.

The invention will be further illustrated by the following non-limiting examples.

### EXAMPLE 1

Expression of OB-Immunoadhesins

Using protein engineering techniques, the human OB protein was expressed as a fusion with the hinge, CH2 and CH3 domains of IgG-1. DNA constructs encoding the chimera of the human OB protein and IgG-1 Fc domains were made with the Fc region clones of human IgG-1. Human OB cDNA was obtained by PCR from human fat cell dscDNA (Clontech Buick-Clone cDNA product). The source of the IgG-1 cDNA was the plasmid pBSSK-CH₂CH₃. The chimera contained the coding sequence of the full length OB protein (amino acids 1-167 in FIG. 5) and human IgG-1 sequences beginning at aspartic acid 216 (taking amino acid 114 as the first residue of the heavy chain constant region (Kabat et al., Sequences of Proteins of Immunological Interest 4th ed. [1987]), which is the first residue of the IgG-1 hinge after the cysteine residue involved in heavy-light chain bonding, and ending with residues 441 to include the CH2 and CH3 Fc domains of IgG-1. There was an insert of codons for three amino acids (GlyValThr) between the OB and IgG-1 coding sequences. If necessary, this short linker sequence can easily be deleted, for example by site directed deletion mutagenesis, to create an exact junction between the coding sequences of the OB protein and the IgG-1 hinge region. The coding sequence of the OB-IgG-1 immunoadhesin was subcloned into the pRK5-based vector pRK5tk-neo which contains a neomycine selectable marker, for transient expression in 293 cells using the calcium phosphate technique (Suva et al., Science 237, 893-896 [1987]). 293 cells were cultured in HAM's: Low Glucose DMEM medium (50:50), containing 10% FBS and 2 mM L-Gln. For purification of OB-IgG-1 chimeras, cells were changed to serum free production medium PS24 the day after transfection and media collected after three days. The culture media was filtered.

The filtered 293 cell supernatant (400 ml) containing recombinant human OB-IgG-1 was made 1 mM in phenylmethylsulfonyl fluoride and 2 μg/ml in aprotinin. This material was loaded at 4° C. onto a 1×4.5 cm Protein A agarose column (Pierce catalog # 20365) equilibrated in 100 mM HEPES pH 8. The flow rate was 75 ml/h. Once the sample was loaded, the column was washed with equilibration buffer until the A₂₈₀ reached baseline. The OB-IgG-1 protein was eluted with 3.5 M MgCl₂+2% glycerol (unbuffered) at a flow rate of 15 ml/h. The eluate was collected with occasional mixing into 10 ml of 100 mM HEPES pH 8 to reduce the MgCl₂ concentration by approximately one-half and to raise the pH. The eluted protein was then dialyzed into phosphate buffered saline, concentrated, sterile filtered and stored either at 4° C. or frozen at −70° C. The OB-IgG-1 immunoadhesin prepared by this method is estimated by SDS-PAGE to be greater than 90% pure.

### EXAMPLE 2

### Animal Studies

A. Materials and Methods

OB protein Production—Murine OB cDNA was obtained by PCR from an adipocyte cDNA library using primers based on the sequence of Zhang et al., supra. Mature OB protein (amino acids 22-167) was expressed in E. coli by inserting the OB coding sequence in frame with the secretion sequence of the E. coli heat-stable enterotoxin II, downstream of the E. coli alkaline phosphatase promoter. Chang et al., Gene 55, 189-96 (1987). After cell lysis, the insoluble fraction was solubilized in 8 M urea buffer pH 8.35 in the presence of 25 mM DTT. Reduced OB protein was purified by size exclusion and reverse phase HPLC, then refolded in the presence of glutathione. Refolded OB protein was purified by reverse phase HPLC and analyzed by SDS-PAGE and amino acid and mass spectrometry analyses.

Preparation of PEG-hOB—The PEG derivatives of the human PB protein were prepared by reaction of hOB purified by reverse phase chromatography with a succinimidyl derivative of PEG propionic acid (SPA-PEG) having a nominal molecular weight of 10 kD, which had been obtained from Shearwater Polymers, Inc. (Huntsville, Ala.). After purification of the hOB protein by reverse phase chromatography, an approximately 1-2 mg/ml solution of the protein in 0.1% trifluoroacetic acid and approximately 40% acetonitrile, was diluted with ⅓ to ½ volume of 0.2 M borate buffer and the pH adjusted to 8.5 with NaOH. SPA-PEG was added to the reaction mixture to make 1:1 and 1:2 molar ratios of protein to SPA-PEG and the mixture was allowed to incubate at room temperature for one hour. After reaction and purification by gel electrophoresis or ion exchange chromatography, the samples were extensively dialyzed against phosphate-buffered saline and sterilized by filtration through a 0.22 micron filter. Samples were stored at 4° C. Under these conditions, the PEG-hOB resulting from the 1:1 molar ratio protein to SPA-PEG reaction consisted primarily of molecules with one 10 kD PEG attached with minor amounts of the 2 PEG-containing species. The PEG-hOB from the 1:2 molar reaction consisted of approximately equal amounts of 2 and 3 PEGs attached to hOB, as determined by SDS gel electrophoresis. In both reactions, small amounts of unreacted protein was also detected. This unreacted protein can be efficiently removed by the gel filtration or ion exchange steps as needed. The PEG derivatives of the human OB protein can also be prepared essentially following the aldehyde chemistry described in EP 372,752 published Jun. 13, 1990.

Animal Studies—All manipulations involving animals were reviewed and approved by Genentech's Institutional Animal Care and Use Committee. Seven to eight week-old genetically obese C57BI/6J-ob/ob (ob/ob) female mice were purchase from Jackson Labs (Bar Harbor, Me.). Lean female mice of the same genetic background (C57BI/6) were purchased from Harlan Sprague Dawley (Hollister, Calif.). Mice were housed in groups 3-6 with ad libitum access to water and standard mouse chow (Purina 5010; Purina Mills, Richmond, Ind.) in a temperature-, humidity- and light-controlled (lights on at 06:00 h, of at 18:00 h) colony room.

Miniosmotic pumps (Alzet model 2002; Alza Corp., Palo Alto, Calif.) were filled with purified recombinant OB protein (100 μg/kg/day) in sterile phosphate-buffered saline (PBS) or PBS alone under sterile conditions following manufacturer's instructions and incubated overnight in sterile saline at room temperature prior to implantation into mice. Mice were anesthetized with ketamine/xylazine, and miniosmotic pumps were implanted subcutaneously in the midscapular region. Daily subcutaneous injections of purified recombinant OB protein, hOB-IgG-1 fusion protein or PBS were made into the midscapular region of conscious mice. Injections were performed within one hour of lights out. The body weight of each mouse (to the nearest 0.1 gram) and the weight of the food contained in the food bin in each cage (to the nearest 0.1 gram) were recorded within one hour of lights out every one to two days. The data are depicted as the mean±SEM. The number of animals is as described below and in the Figure legends.

B. Results with Continuous Subcutaneous Infusion of OB Protein

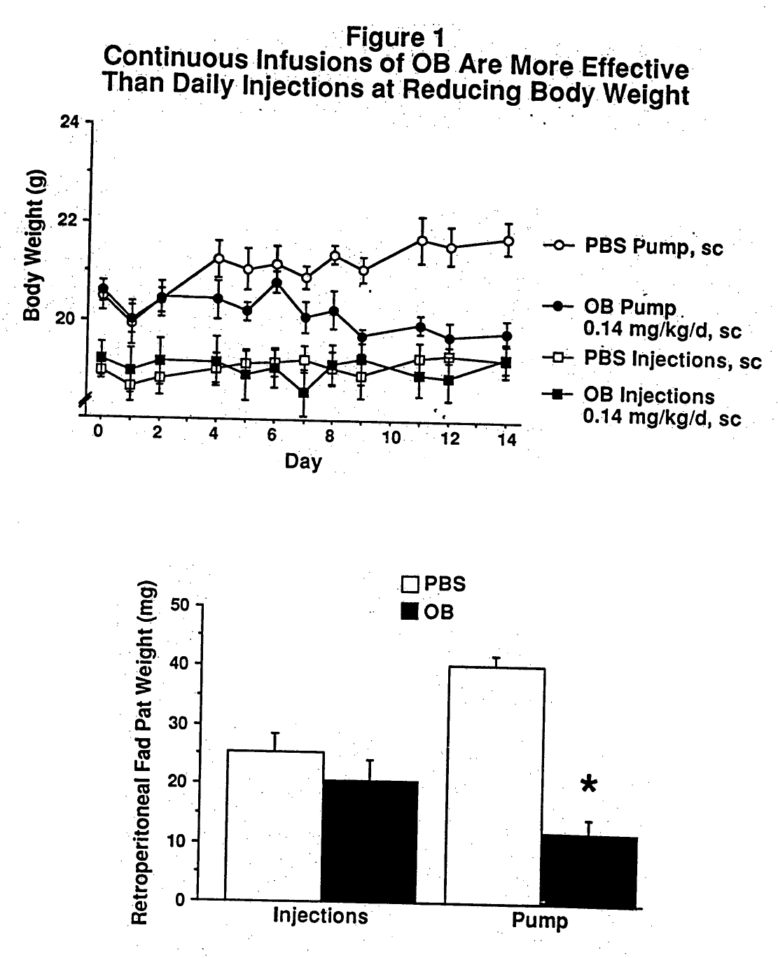

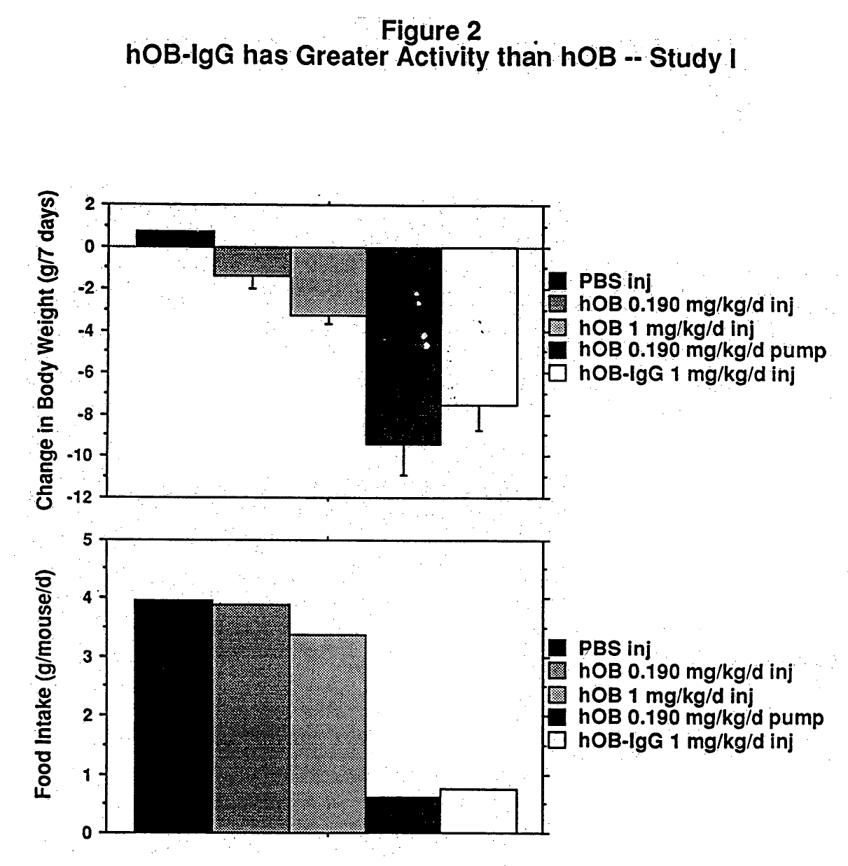

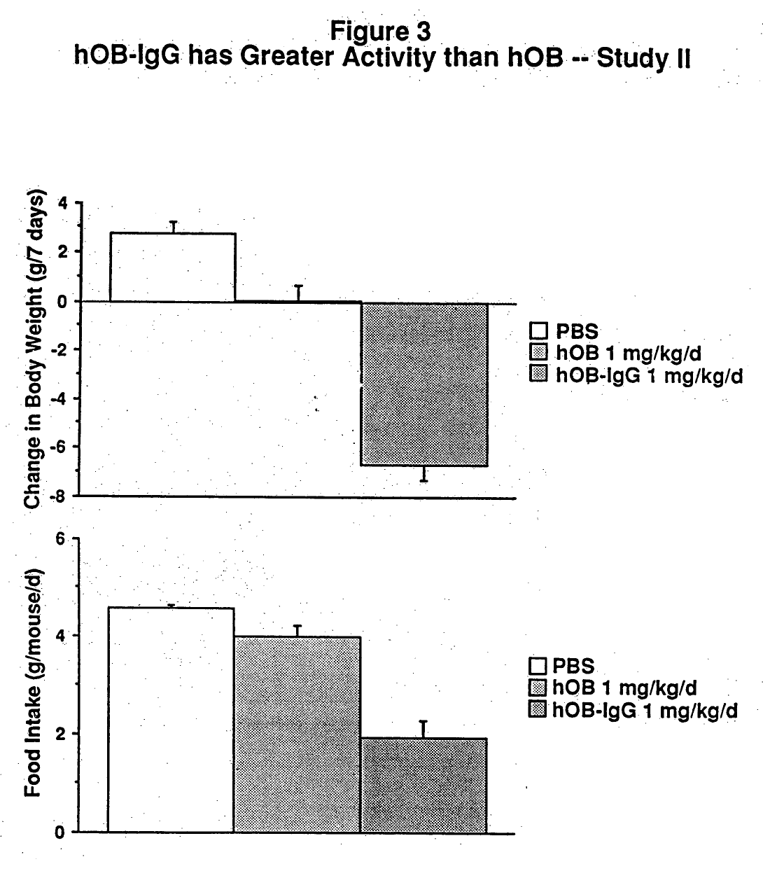

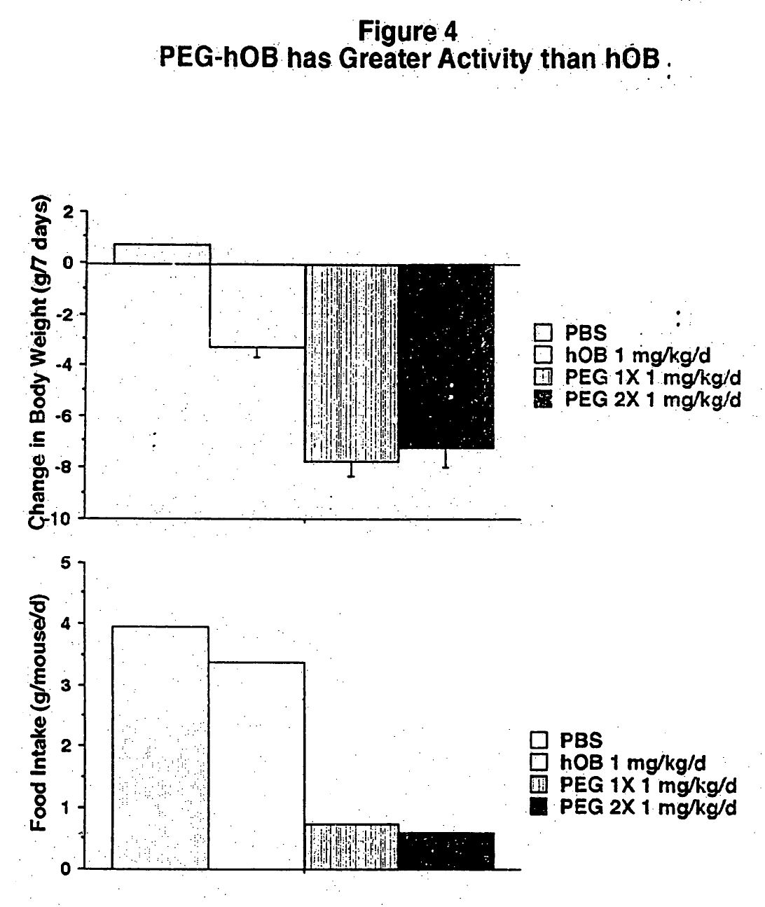

Lean female mice were treated with murine OB protein either as a continuous subcutaneous infusion or daily subcutaneous injections. The results are shown in FIG. 1. The upper chart shows that the OB protein is significantly more effective in reducing body weight when delivered as a continuous infusion than when the same dose is delivered in the form of daily subcutaneous injections. The bottom chart shows the same difference in the ability of the OB protein to reduce adipose tissue weight.

C. Results with the OB-IgG-1 Chimera

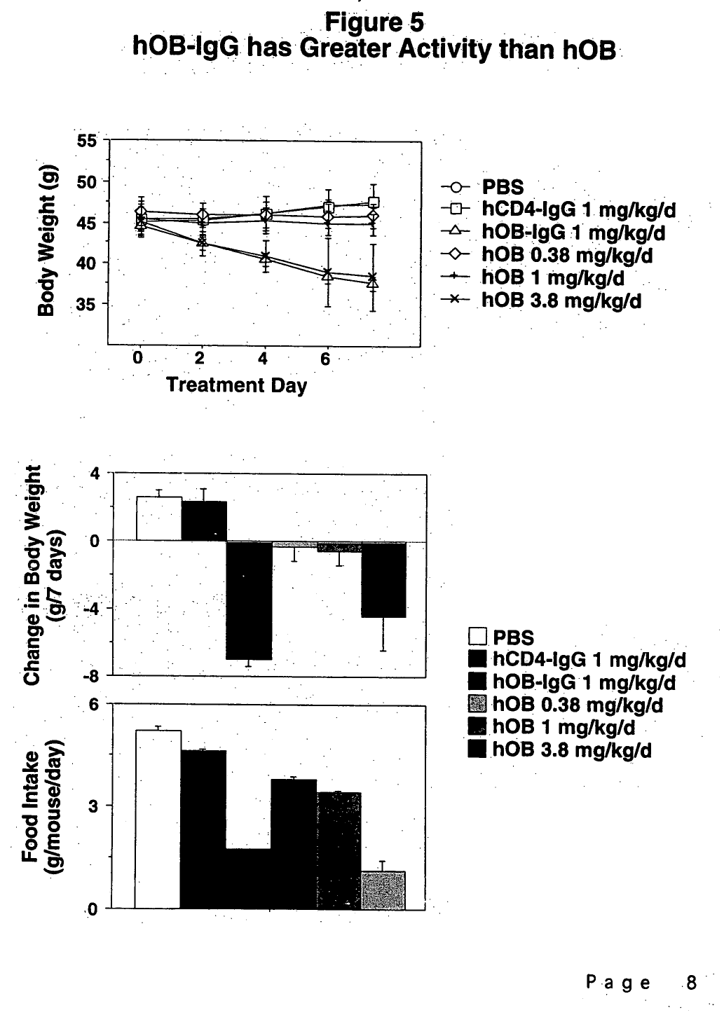

Obese female ob/ob mice were treated with human OB protein or with the human OB-IgG-1 chimera. The data are shown in FIG. 2. The data presented in the top chart demonstrate that the hOB-IgG-1 fusion protein is more potent than the native hOB protein at reducing body weight, when both proteins are administered similarly by daily subcutaneous infusion. It is noted that the increase in potency would be even more expressed, if the data were converted to molar amounts, as only about one third of the OB-IgG-1 chimera comes from the OB protein. The data further confirm the previous observation that continuous subcutaneous infusion (pump) or the hOB protein is more effective than daily subcutaneous injections (inj) at reducing body weight.

The data shown at the bottom chart of FIG. 2 show that the hOB-IgG-1 fusion protein substantially reduced food intake. This result was unexpected as it was assumed that the fusion protein would be too large to cross the blood-brain barrier and exert its effect.

Obese (ob/ob) female mice were treated with either hOB or the hOB-IgG-1 chimera by daily subcutaneous injections for 7 days. The data shown in FIG. 3 again demonstrate that the chimera is more effective than the native hOB protein at reducing body weight (top) and food intake (bottom).

In a further experiment, obese (ob/ob) female mice were treated with either the hOB-IgG-1 fusion protein, native hOB or hCD4-IgG-1 (control) by daily subcutaneous injections for seven days. The results shown in FIG. 5 affirm that the hOB-IgG-1 fusion protein is more effective than the native hOB protein at reducing body weight (top and middle panels) and food intake (bottom panel).

D. Results with PEG-hOB

Obese female ob/ob mice were treated with human OB protein or with PEG derivatives of human OB. The data are shown in FIG. 4. The data presented in the top chart demonstrate that PEG-hOB is more potent than the native hOB protein at reducing body weight, when both proteins are administered similarly by daily subcutaneous infusion.

The data shown at the bottom chart of FIG. 4 show that the PEG-hOB proteins were substantially more effective in reducing food intake than unmodified native hOB.

### REFERENCE EXAMPLE

Identification and Cloning of an OB Receptor

The OB protein-immunoadhesin of Example 1 was used to detect and expression clone an OB receptor. First, to identify a receptor source, several cell lines were screened with 1 μg/ml OB-IgG-1 fusion by flow cytometry. The detection system which consists of a biotin conjugated secondary antibody followed by streptavidin-phycoerythrin provides a dramatic amplification of the signal and allows the detection of cells expressing low numbers of receptors. Two cell lines, human embryonic kidney 293 and human lung A549 cells were found to bind OB-IgG-1 but not an Flt-4 control immunoadhesin. Specific binding of OB-IgG-1 to the cells was also demonstrated by the addition of excess of bacterially expressed human OB protein. Addition of 10 μg/ml of human OB completely blocks the binding of OB-IgG-1 to 293 cells.

To isolate a cDNA encoding the OB receptor, COSN cells were transiently transfected with pools of approximately 10⁵ clones of an oligo dT primed 293 cell cDNA library in pRK5B. Transfected cells were enriched by panning on plates coated with an anti-human Fc antibody after incubation with OB-IgG-1. After three rounds of enrichment, 1 of 30 pools gave rise to OB-IgG-1 mediated adherence of COSN cells to the binding plates which could be competed by human leptin. cDNA clones picked randomly from this third round were transfected in pools of 10-20. Individual clones were finally identified after breaking down one pool of 10 that was scoring positive by panning.

Sequence analysis revealed a clone of approximately 5300 bp with an open reading frame encoding a protein of 896 amino acids. The sequence corresponded to a type I transmembrane protein with a 22 amino acid long signal peptide, 819 amino acid extracellular domain, 21 amino acid transmembrane domain and a short 34 amino acid intracellular domain. The sequence was found to essentially correspond to the human OB receptor identified and isolated by Tartaglia et al., supra, and is identical with a human receptor sequence disclosed in copending application Ser. No. 08/585,005 filed Jan. 11, 1996.

While the invention has been illustrated by way of examples, the scope of the invention is not so limited. It will be understood that further modifications and variations are possible without diverting from the overall concept of the invention. All such modifications are intended to be within the scope of the present invention.

All references cited throughout the specification, including the examples, and the references cited therein are hereby expressly incorporated by reference.

What is claimed is:

1. A long half-life derivative of an OB protein retaining a biological property of a native OB protein.

2. The long half-life derivative of claim 1 capable of reducing body weight and/or food intake in an individual treated.

3. The derivative of claim 1 which is a derivative of a native human OB protein.

4. The derivative of claim 1 which is an OB-immunoglobulin chimera.

5. The derivative of claim 1 which is a native OB protein or an OB-immunoglobulin chimera modified with a nonproteinaceous polymer.

6. The derivative of claim 5 wherein the nonproteinaceous polymer is polyethylene glycol (PEG).

7. A composition for the treatment of a condition associated with the abnormal expression or function of the OB gene, or for eliciting a biological response mediated by an OB receptor, comprising an effective amount of an OB derivative of claim 1.

8. The composition of claim 7 effective for weight and/or appetite reduction.

9. The composition of claim 7 effective in the reduction of elevated insulin levels.

10. A method for the treatment of a condition associated with the abnormal expression or function of the OB gene, or for eliciting a biological response mediated by an OB receptor, comprising administering to an individual to be treated a derivative of claim 1.

11. The method of claim 10 wherein the condition to be treated is selected from the group consisting of obesity, bulimia, and Type I or II diabetes.

12. A method for inducing weight loss or appetite loss in a subject, comprising administering to said subject an effective amount of a derivative of claim 1.

13. A chimeric polypeptide comprising an OB protein amino acid sequence capable of binding to a native OB receptor, linked to an immunoglobulin sequence.

14. The chimeric polypeptide of claim 13 wherein said immunoglobulin sequence is a constant domain sequence.

15. The chimeric polypeptide of claim 14 wherein said OB protein is human.

16. The chimeric polypeptide of claim 15 wherein two OB polypeptide-IgG heavy chain fusions are linked to each other by at least one disulfide bond to yield a homodimeric immunoglobulin-like structure.

17. The chimeric polypeptide of claim 16 wherein at least one of said OB polypeptide-IgG heavy chain fusions is associated with an immunoglobulin light chain.

18. An isolated nucleic acid sequence encoding an OB protein-immunoglobulin fusion.

19. A replicable expression vector comprising the nucleic acid of claim 18.

20. A host cell transformed wih the replicable expression vector of claim 19.

21. A process comprising culturing the host cells of claim 20 so as to express the nucleic acid encoding an OB protein-immunoglobulin fusion.

22. The process of claim 21 wherein said host cells are cotransformed with nucleic acid encoding at least two OB protein-immunoglobulin fusions.

23. The process of claim 22 wherein said cells are further transformed with nucleic acid encoding at least one immunoglobulin light chain.

24. A method of treating a condition associated with the abnormal expression or function of the OB gene or for eliciting a biological response mediated by an OB receptor comprising administering to a patient a therapeutically effective amount of the chimeric polypeptide of claim 13.

25. The method of claim 24 wherein said condition is selected from the group consisting of obesity, bulimia and type I or II diabetes.

26. A composition for the treatment of obesity comprising an effective amount of a chimeric polypeptide of claim 13 in association with a pharmaceutically acceptable carrier.

27. A method for inducing the growth of cells expressing an OB receptor comprising contacting said cells with the OB derivative of claim 1.
